(19) United States
(12) Patent Application Publication
(10) Pub. No.: US 2024/0074836 A1
(43) Pub. Date: Mar. 7, 2024

(54) DEVICES FOR TREATING TEETH AND ASSOCIATED SYSTEMS AND METHODS

(71) Applicant: Brius Technologies, Inc., Carrollton, TX (US)

(72) Inventors: Seyed Mehdi Roein Peikar, Carrollton, TX (US); James Sylvester Wratten, Jr., Waterville, NY (US); Woojae Kim, Addison, TX (US)

(21) Appl. No.: 18/261,822

(22) PCT Filed: Jan. 21, 2022
(86) PCT No.: PCT/US2022/013366

§ 371 Date: Jul. 17, 2023

Related U.S. Application Data
(60) Provisional application No. 63/199,763, filed on Jan. 22, 2021.
(60) Provisional application No. 63/200,723, filed on Mar. 24, 2021.

(51) Int. Cl. A61C 7/22; A61C 7/14

(52) U.S. Cl. CPC A61C 7/22; A61C 7/145; A61C 2201/002

(57) ABSTRACT

Orthodontic devices and associated systems and methods are disclosed herein. In some embodiments, an orthodontic device comprises a marking region configured to facilitate the design, manufacturing, production, distribution, installation, and/or use of the device. The marking region can comprise indicia configured to communicate information regarding the device, an associated patient, an instruction for installation of the device, etc. The indicia can comprise a recess or opening in the device, a raised or protruding portion of the device, and/or material that has been added to the device. In various embodiments, the indicia include, for example, letters, numbers, symbols, colors, shapes, and/or patterns that directly provide and/or encode the information of interest. The indicia can be human-readable and/or machine-readable.

### CROSS-REFERENCE TO RELATED APPLICATION(S)

The present application claims the benefit of priority to U.S. Provisional Patent Application No. 63/199,763, titled DEVICES FOR TREATING TEETH AND ASSOCIATED SYSTEMS AND METHODS, filed Jan. 22, 2021, and to U.S. Provisional Patent Application No. 63/200,723, titled DEVICES FOR TREATING TEETH AND ASSOCIATED SYSTEMS AND METHODS, filed Mar. 24, 2021, each of which is incorporated by reference herein in its entirety.

This application is related to the following applications, each of which is hereby incorporated by reference in its entirety: U.S. patent application Ser. No. 16/865,323, titled DENTAL APPLIANCES, SYSTEMS AND METHODS, filed May 2, 2020; International Patent Application No. PCT/US20/31211, titled DENTAL APPLIANCES, SYSTEMS AND METHODS, filed May 2, 2020; U.S. patent application Ser. No. 15/929,443, titled DENTAL APPLIANCES AND ASSOCIATED SYSTEMS AND METHODS OF USE, filed May 2, 2020; U.S. patent application Ser. No. 15/929,444, titled DENTAL APPLIANCES AND ASSOCIATED SYSTEMS AND METHODS OF USE, filed May 2, 2020, International Patent Application No. PCT/US20/70017, titled DENTAL APPLIANCES AND ASSOCIATED SYSTEMS AND METHODS OF USE, filed May 2, 2020; U.S. patent application Ser. No. 15/929,442, titled DENTAL APPLIANCES AND ASSOCIATED METHODS OF MANUFACTURING, filed May 2, 2020, International Patent Application No. PCT/US20/70016, titled DENTAL APPLIANCES AND ASSOCIATED METHODS OF MANUFACTURING, filed May 2, 2020, U.S. Provisional Patent Application No. 62/956,290, filed Jan. 1, 2020, U.S. Provisional Patent Application No. 62/842,391, filed May 2, 2019, and U.S. Provisional Patent Application No. 62/704,545, titled DENTAL APPLIANCES AND ASSOCIATED SYSTEMS AND METHODS OF USE, filed May 15, 2020.

### TECHNICAL FIELD

The present technology relates to orthodontic devices for treating teeth and associated systems and methods.

### BACKGROUND

A common objective in orthodontics is to move a patient's teeth to positions where the teeth function optimally and aesthetically. To move the teeth, the orthodontist may begin by obtaining multiple scans and/or impressions of the patient's teeth to determine a series of corrective paths between the initial positions of the teeth and the desired ending positions. The orthodontist then fits the patient to one of two main appliance types: braces or aligners.

Traditional braces consist of brackets and an archwire placed across a front side of the teeth, with elastic ties or ligature wires to secure the archwire to the brackets. In some cases self-ligating brackets may be used in lieu of ties or wires. The shape and stiffness of the archwire as well as the archwire-bracket interaction governs the forces applied to the teeth and thus the direction and degree of tooth movement. To exert a desired force on the teeth, the orthodontist often manually bends the archwire. The orthodontist monitors the patient's progress through regular appointments, during which the orthodontist visually assesses the progress of the treatment and makes manual adjustments to the archwire (such as new bends) and/or replaces or repositions brackets. The adjustment process is both time consuming and tedious for the patient and more often than not results in patient discomfort for several days following the appointment. Moreover, braces are not aesthetically pleasing and make brushing, flossing, and other dental hygiene procedures difficult.

Aligners comprise clear, removable, polymeric shells having cavities shaped to receive and reposition teeth to produce a final tooth arrangement. Aligners offer patients significantly improved aesthetics over braces. Aligners do not require the orthodontists to bend wires or reposition brackets and are generally more comfortable than braces. However, unlike braces, aligners cannot effectively treat all malocclusions. Certain tooth repositioning steps, such as extrusion, translation, and certain rotations, can be difficult or impossible to achieve with aligners. Moreover, because the aligners are removable, success of treatment is highly dependent on patient compliance, which can be unpredictable and inconsistent.

Lingual braces are an alternative to aligners and traditional (buccal) braces and have been gaining popularity in recent years. Two examples of existing lingual braces are the Incognito™ Appliance System (3M United States) and INBRACE® (Swift Health Systems, Irvine, California, USA), each of which consists of brackets and an archwire placed on the lingual, or tongue side, of the teeth. In contrast to traditional braces, lingual braces are virtually invisible, and, unlike aligners, lingual braces are fixed to the patient's teeth and force compliance. These existing lingual technologies, however, also come with several disadvantages. Most notably, conventional lingual appliances still rely on a bracket-archwire system to move the teeth, thus requiring multiple office visits and painful adjustments. For example, lingual technologies have a relatively short inter-bracket distance, which generally makes compliance of the archwire stiffer. As a result, the overall lingual appliance is more sensitive to archwire adjustments and causes more pain for the patient. Moreover, the lingual surfaces of the appliance can irritate the tongue and impact speech, and make the appliance difficult to clean.

Therefore, a need exists for improved orthodontic appliances.

### SUMMARY

The subject technology is illustrated, for example, according to various aspects described below, including with reference to FIGS. 1-17. Various examples of aspects of the subject technology are described as numbered clauses (1, 2, 3, etc.) for convenience. These are provided as examples and do not limit the subject technology.

1. An orthodontic appliance configured to be secured to a patient's teeth, the appliance comprising:
a resilient member configured to extend along the patient's teeth and to reposition the patient's teeth from an initial position to a desired position, the resilient member comprising one or more securing portions configured to be secured to one or more of the teeth and a marker including indicia configured to communicate information corresponding to at least one of an identity of the patient, an intended position of the appliance relative to the teeth once installed, or an origin of the appliance.

2. The appliance of Clause 1, wherein the indicia comprise a recess or opening in the resilient member.

3. The appliance of any one of the preceding Clauses, wherein the information corresponding to the origin of the appliance includes a serial number, a stocking keeping unit, and/or a lot number.

4. The appliance of any one of the preceding Clauses, wherein the information corresponding to the identity of the patient comprises a combination of at least one of numbers, letters, symbols, shapes, colors, or patterns, and wherein the combination is unique to the patient.

5. The appliance of any one of the preceding Clauses, wherein the information corresponding to the intended position of the appliance relative to the teeth indicates which of the patient's teeth the appliance is configured to be secured to.

6. The appliance of any one of the preceding Clauses, wherein the indicia include at least one of numbers, letters, symbols, colors, shapes, or patterns.

7. The appliance of any one of the preceding Clauses, wherein the indicia is human-readable and/or machine readable.

8. The appliance of any one of the preceding Clauses, wherein the resilient member comprises a metal.

9. The appliance of any one of the preceding Clauses, wherein the resilient member is formed from a wire and/or a sheet of material.

10. An orthodontic appliance configured to be secured to a patient's teeth, the appliance comprising:
an anchor configured to extend along the patient's teeth, the anchor including a marking region configured to communicate information corresponding to at least one of an identity of the patient, an intended position of the appliance relative to the teeth once installed, or an origin of the appliance; and
a plurality of arms protruding from and unitarily formed with the anchor, each of the arms including—
   a spring portion configured to apply a biasing force to one of the teeth, and
   a connector portion distal to the spring portion, wherein the connector portion is configured to be coupled to the one of the teeth.

11. The appliance of Clause 10, wherein the marking region includes indicia comprising a recess in the anchor.

12. The appliance of any one of the preceding Clauses, wherein the marking region includes indicia comprising an opening extending through an entire thickness of the anchor.

13. The of appliance of any one of the preceding Clauses, wherein the marking region comprises indicia including at least one of a number, a letter, a symbol, a color, a shape, or a pattern.

14. The appliance of any one of the preceding Clauses, wherein the marking region comprises indicia including a unique combination of numbers, letters, symbols, colors, shapes, and/or patterns encoding an identity of the specific patient.

15. The appliance of any one of the preceding Clauses, wherein the marking region comprises indicia defined by at least one of a protrusion, a recess, or an opening.

16. The appliance of any one of the preceding Clauses, wherein the marking region comprises indicia formed by at least one of an ink, a film, a coating, or a surface treatment of the appliance.

17. The appliance of any one of the preceding Clauses, wherein the marking region comprises an RFID tag.

18. The appliance of any one of the preceding Clauses, wherein the marking region is human-readable.

19. The appliance of any one of the preceding Clauses, wherein the marking region is machine-readable.

20. The appliance of any one of the preceding Clauses, wherein the marking region includes indicia positioned at a lingual-facing side of the anchor.

21. The appliance of any one of the preceding Clauses, wherein the marking region includes indicia positioned at a facial-facing side of the anchor.

22. The appliance of any one of the preceding Clauses, wherein the information corresponding to an intended position of the appliance with respect to the patient's teeth includes an indication of which dental arch of the patient the appliance is configured to be secured to and/or an indication of which of the patient's teeth the appliance is configured to be secured to.

23. The appliance of any one of the preceding Clauses, wherein the information corresponding to the origin of the appliance is at least one of a serial number, a lot number, or a stock keeping unit.

24. The appliance of any one of the preceding Clauses, wherein the marking region is a first marking region, the anchor further comprising a second marking region configured to communicate information corresponding to at least one of an identity of the patient, an intended position of the appliance relative to the teeth once installed, or an origin of the appliance.

25. The appliance of any one of the preceding Clauses, wherein the marking region includes permanent indicia.

26. The appliance of any one of the preceding Clauses, wherein, when the appliance is secured to the patient's teeth, the marking region is not visible within an oral cavity of the patient to a person directly viewing the patient's oral cavity.

27. The appliance of any one of the preceding Clauses, wherein a first width of the anchor at or adjacent to the marking region is greater than a second width of the anchor away from the marking region.

28. An orthodontic appliance configured to be secured to a patient's teeth, the appliance comprising:
an anchor configured to be disposed along the patient's teeth;
a plurality of arms extending from the anchor, each of the arms including (i) a first end portion extending from the anchor, (ii) a free second end portion, (iii) a connector portion configured to be coupled to one of the teeth, and (iv) a spring portion configured to apply a biasing force to at least one of the teeth, the spring portion disposed along the arm between the first end portion and the connector portion, wherein the anchor and at least some of the arms comprise a three-dimensional unitarily-formed structure; and
a marker configured to communicate information corresponding to at least one of an identity of the patient, an intended position of the appliance relative to the teeth once installed, or an origin of the appliance.

29. The appliance of Clause 28, wherein the information corresponding to an intended position of the appliance with respect to the patient's teeth includes an indication of which of the patient's teeth the appliance is configured to be secured to.

30. The appliance of any one of the preceding Clauses, wherein the information corresponding to an intended position of the appliance with respect to the patient's teeth includes an indication of which dental arch of the patient the appliance is configured to be secured to.

31. The appliance of any one of the preceding Clauses, wherein the marker comprises at least one of a number, a letter, a symbol, a color, a shape, or a pattern.

32. The appliance of any one of the preceding Clauses, wherein the marker comprises at least one of a protrusion, a recess, or an opening.

33. The appliance of any one of the preceding Clauses, wherein the marker comprises at least one of an ink, a film, a coating, or a surface treatment of the appliance.

34. The appliance of any one of the preceding Clauses, wherein the marker is human-readable.

35. The appliance of any one of the preceding Clauses, wherein the marker is machine-readable.

36. The appliance of any one of the preceding Clauses, wherein the marker is positioned at the anchor.

37. The appliance of Clause 36, wherein the marker is positioned at or adjacent to a midline of the anchor.

38. The appliance of any one of the preceding Clauses, wherein the marker is positioned at one or more of the plurality of arms.

39. The appliance of any one of the preceding Clauses, wherein the marker is positioned at a lingual-facing side of the appliance.

40. The appliance of any one of the preceding Clauses, wherein the marker is positioned at a facial-facing side of the appliance.

41. The appliance of any one of the preceding Clauses, wherein the marker is a first marker, the appliance further comprising a second marker that associates the appliance with a specific patient.

42. The appliance of Clause 41, wherein the second marker is spaced apart from the first marker.

43. The appliance of any one of the preceding Clauses, wherein the marker is permanent.

44. The appliance of any one of the preceding Clauses, wherein, when the appliance is secured to the patient's teeth, the marker is not visible within an oral cavity of the patient to a person directly viewing the patient's oral cavity.

45. An orthodontic appliance configured to be secured to a patient's teeth, the appliance comprising:
an elongate member configured to extend along the patient's teeth;
a plurality of arms cantilevered from, spaced apart along, and unitarily formed with the elongate member, each of the arms including—
   a first portion configured to be coupled to a corresponding one of the teeth, and
   a second portion proximal of the first portion along the respective one of the arms, wherein the second portion is resiliently flexible such that, when the first portion is coupled to the corresponding one of the teeth, the second portion is configured to move from a loaded state toward an unloaded state, thereby repositioning the corresponding one of the teeth from an original position toward a desired final position,
first indicia providing information that associates the appliance with a specific patient; and
second indicia providing information corresponding to an intended position and orientation of the appliance with respect to the patient's teeth.

46. A method of manufacturing an orthodontic appliance configured to be secured to a patient's teeth, the method comprising:
forming a substantially planar member based on planar shape data for the appliance;
manipulating the member into a 3D configuration;
shape-setting the member in the 3D configuration; and
modifying the member to comprise a marking region including indicia configured to communicate information corresponding to at least one of an origin of the appliance, an identity of the patient, or an instruction for installing the appliance.

47. The method of Clause 46, wherein modifying the member comprises creating one or more recesses in the member via at least one of laser cutting, laser engraving, milling, wire electrical discharge machining, water jetting, etching, punching, or stamping.

48. The method of Clause 47, wherein the one or more recesses extend completely through a thickness of the appliance.

49. The method of any one of the preceding Clauses, wherein modifying the member comprises depositing one or more materials on a surface of the member via at least one of vacuum deposition, additive manufacturing, electroplating, printing, or coating.

50. The method of any one of the preceding Clauses, wherein modifying the member comprises creating one or more raised portions in the member via at least one of embossing, milling, or machining.

51. The method of any one of the preceding Clauses, wherein modifying the member comprises coupling the indicia to the member via at least one of welding, adhesive bonding, or soldering.

52. The method of any one of the preceding Clauses, wherein the information corresponding to the origin of the appliance includes a serial number, a stocking keeping unit, and/or a lot number.

53. The method of any one of the preceding Clauses, wherein the information corresponding to the identity of the patient comprises a combination of at least one of numbers, letters, symbols, colors, shapes, or patterns, and wherein the combination is unique to the patient.

54. The method of any one of the preceding Clauses, wherein the information corresponding to the instruction for installing the appliance indicates an intended position and/or orientation of the appliance with respect to the patient's teeth.

55. The method of any one of the preceding Clauses, wherein the instruction for installing the appliance indicates which of the patient's teeth the appliance is configured to be secured to.

56. The method of any one of the preceding Clauses, wherein the indicia includes a combination of at least one of numbers, letters, symbols, colors, shapes, or patterns.

57. The method of any one of the preceding Clauses, wherein the indicia is human-readable.

58. The method of any one of the preceding Clauses, wherein the indicia is machine-readable.

59. The method of any one of the preceding Clauses, wherein the indicia and member comprise a monolithic structure.

60. A method of manufacturing an orthodontic appliance configured to be secured to a patient's teeth, the method comprising:
cutting a substantially planar member from a sheet of material;
while cutting the member from the sheet of material, removing material from the member to form indicia configured to communicate information corresponding to at least one of an origin of the appliance, an identity of the patient, or an instruction for installing the appliance;
manipulating the member into a 3D configuration; and
shape-setting the member in the 3D configuration, thereby forming the orthodontic appliance for installation.

61. The method of any one of the preceding Clauses, wherein the one or more recesses extend completely through a thickness of the appliance.

62. The method of any one of the preceding Clauses, wherein the indicia includes a combination of at least one of numbers, letters, symbols, colors, shapes, or patterns.

63. The method of any one of the preceding Clauses, wherein the indicia is machine-readable.

64. An orthodontic device comprising:
a body portion configured to be secured to one or more of a patient's teeth; and
a marking portion comprising a first portion spaced apart from the body portion and a second portion disposed between and connecting the first portion of the marking portion and the body portion, the second portion having a width less than a width of the first portion, wherein the first portion includes indicia providing information corresponding to at least one of an identity of the patient, an intended position of the device relative to the teeth once installed, or an origin of the device.

65. The device of any one of the preceding Clauses, wherein the indicia comprises a letter, a number, a symbol, and/or a shape.

66. The device of any one of the preceding Clauses, wherein the first portion of the marking portion has a first shape and the indicia has a second shape that is substantially the same as the first shape.

67. The device of any one of the preceding Clauses, wherein the first portion of the marking portion has a first shape and the indicia has a second shape that is different from the first shape

68. The device of any one of the preceding Clauses, wherein the first shape is substantially rectangular.

69. The device of any one of the preceding Clauses, wherein the second portion has a thickness that is less than a thickness of the first portion.

70. The device of any one of the preceding Clauses, wherein the indicia comprises a marking on the marking portion.

71. The device of any one of the preceding Clauses, wherein the indicia comprises a recess or opening in the marking portion.

72. The device of any one of the preceding Clauses, wherein the indicia comprises a raised region of the marking portion.

73. The device of any one of the preceding Clauses, wherein the second portion is structurally weakened.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIGS. 1A and 1B schematically illustrate directional references relative to a patient's dentition.

FIG. 2A shows a schematic representation of an orthodontic appliance in accordance with the present technology installed in a patient's mouth adjacent the patient's dentition.

FIG. 2B is a schematic depiction of connection configuration options in accordance with the present technology.

FIG. 2C is a schematic depiction of a portion of an appliance in accordance with the present technology.

FIGS. 3A and 3B are elevation views of an appliance in accordance with the present technology installed in an upper and lower jaw of a patient's mouth with the patient's teeth in an original tooth arrangement and a final tooth arrangement, respectively.

FIG. 3C depicts example stress-strain curves of nitinol and steel.

FIG. 4 depicts an orthodontic appliance in accordance with the present technology.

FIGS. 5A and 5B depict a marking region of an orthodontic appliance in accordance with the present technology.

FIG. 6 depicts an orthodontic appliance in accordance with the present technology.

FIG. 7 depicts an orthodontic appliance in accordance with the present technology.

FIG. 8 depicts an orthodontic appliance in accordance with the present technology.

FIG. 9 depicts an orthodontic appliance in accordance with the present technology.

FIG. 10 depicts an orthodontic appliance in accordance with the present technology.

FIG. 11 depicts an orthodontic appliance in accordance with the present technology.

FIG. 12 depicts an orthodontic appliance in accordance with the present technology.

FIG. 13 depicts a marking region of an orthodontic appliance in accordance with the present technology.

FIG. 14 depicts a marking region of an orthodontic appliance in accordance with the present technology.

FIG. 15 depicts an orthodontic device in accordance with the present technology.

FIG. 16 depicts an orthodontic device in accordance with the present technology.

FIG. 17 depicts an orthodontic appliance in accordance with the present technology.

### DETAILED DESCRIPTION

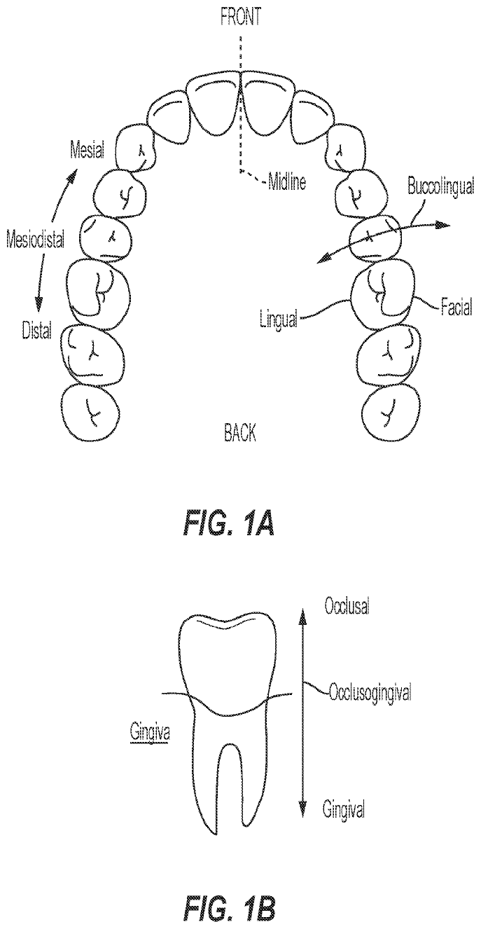

The present technology relates to orthodontic appliances. Various embodiments of the present technology, for example, are directed to orthodontic appliances including a marking region that is configured to facilitate the design, production, distribution, installation, and/or use of the appliance without substantially affecting patient comfort. In some embodiments, the marking region includes indicia configured to communicate information regarding the appliance, the patient, installation of the appliance, etc. For example, the marking region can include indicia associating the appliance with a specific patient. Such information can facilitate appliance tracking during manufacturing and distribution of the appliance. Moreover, such information can be used by an operator installing the appliance in the patient's mouth to ensure that the appliance is installed into the correct patient's mouth. In some embodiments, an appliance includes a marking region that is configured to facilitate proper positioning of the appliance when installed into a patient's mouth. Such marking region can comprise indicia configured to communicate which of the patient's teeth the appliance is configured to be secured to, an intended position or orientation of the appliance with respect to the patient's teeth, etc. In some embodiments, an appliance comprises a marking region comprising indicia configured to communicate an origin of the appliance, such as a serial number, a lot number, or a stock keeping unit (SKU) of the appliance to facilitate tracking and traceability of the appliance throughout manufacturing, distribution, and/or use of the appliance. According to various embodiments of the present technology, the indicia can be human-readable and/or machine readable. The indicia may include one or more numbers, letters, symbols, colors, shapes, patterns, and/or unique or identifiable portions of the appliance to directly provide and/or encode information. Specific details of several embodiments of the technology are described below with reference to FIGS. 1-17.

### I. Definitions

FIGS. 1A and 1B schematically depict several directional terms related to a patient's dentition. Terms used herein to provide anatomical direction or orientation are intended to encompass different orientations of the appliance as installed in the patient's mouth, regardless of whether the structure being described is shown installed in a mouth in the drawings. As illustrated in FIGS. 1A and 1B: “mesial” means in a direction toward the midline of the patient's face along the patient's curved dental arch; “distal” means in a direction away from the midline of the patient's face along the patient's curved dental arch; “occlusal” means in a direction toward the chewing surfaces of the patient's teeth; “gingival” means in a direction toward the patient's gums or gingiva; “facial” means in a direction toward the patient's lips or cheeks (used interchangeably herein with “buccal” and “labial”); and “lingual” means in a direction toward the patient's tongue.

As used herein, the terms “proximal” and “distal” refer to a position that is closer and farther, respectively, from a given reference point. In many cases, the reference point is a certain connector, such as an anchor, and “proximal” and “distal” refer to a position that is closer and farther, respectively, from the reference connector along a line passing through the centroid of the cross-section of the portion of the appliance branching from the reference connector.

As used herein, the terms “generally,” “substantially,” “about,” and similar terms are used as terms of approximation and not as terms of degree, and are intended to account for the inherent variations in measured or calculated values that would be recognized by those of ordinary skill in the art.

As used herein, the term “operator” refers to a clinician, practitioner, technician or any person or machine that designs and/or manufactures an orthodontic appliance or portion thereof, and/or facilitates the design and/or manufacture of the appliance or portion thereof, and/or any person or machine associated with installing the appliance in the patient's mouth and/or any subsequent treatment of the patient associated with the appliance.

As used herein, the term “force” refers to the magnitude and/or direction of a force, a torque, or a combination thereof.

### II. Overview of Orthodontic Appliances of the Present Technology

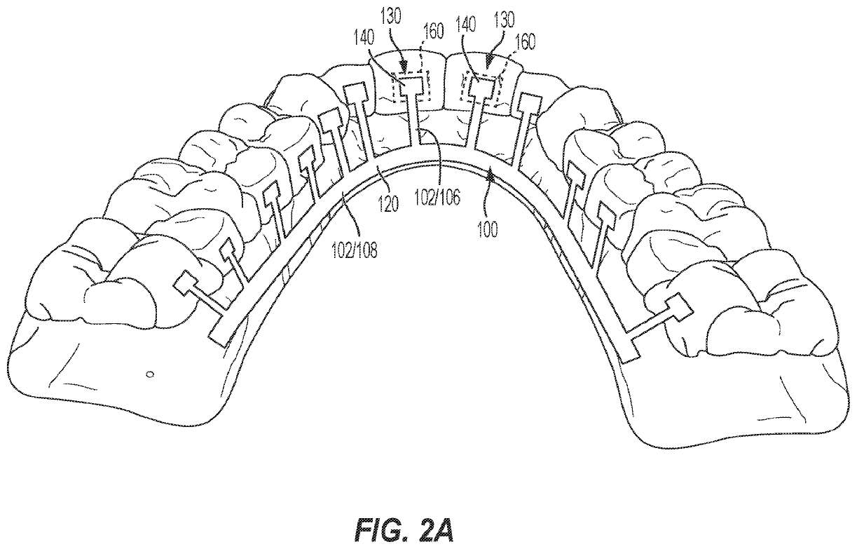

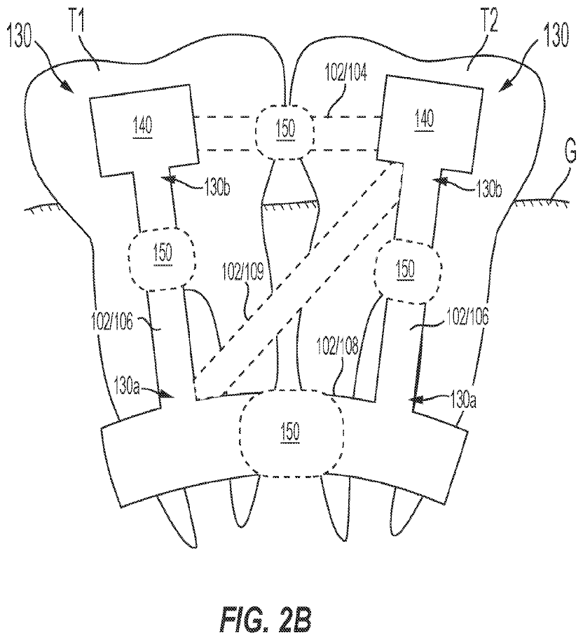

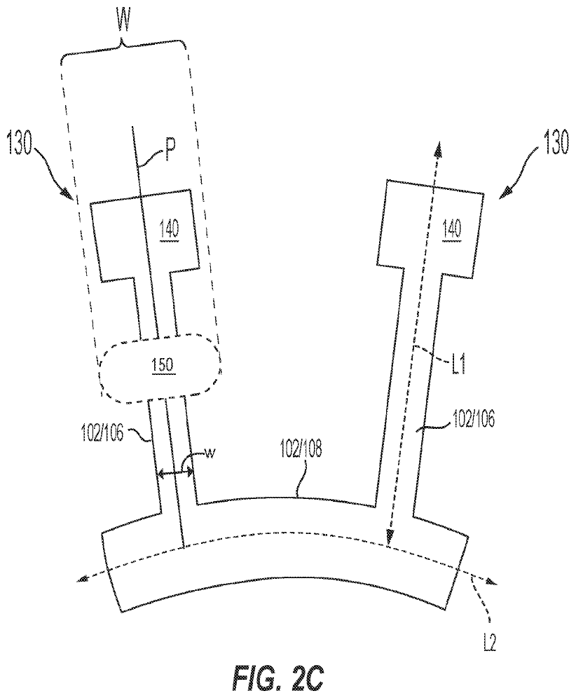

FIG. 2A is a schematic representation of an orthodontic appliance 100 (or “appliance 100”) configured in accordance with embodiments of the present technology, shown positioned in a patient's mouth adjacent the patient's teeth. FIG. 2B is an enlarged view of a portion of the appliance 100. The appliance 100 is configured to be installed within a patient's mouth to impart forces on one or more of the teeth to reposition all or some of the teeth. In some cases, the appliance 100 may additionally or alternatively be configured to maintain a position of one or more teeth. As shown schematically in FIGS. 2A and 2B, the appliance 100 can comprise a deformable member that includes one or more attachment portions 140 (each represented schematically by a box), each configured to be secured to a tooth surface directly or indirectly via a securing member 160. The appliance 100 may further comprise one or more connectors 102 (also depicted schematically), each extending directly between attachment portions 140 (“first connectors 104”), between an attachment portion 140 and one or more other connectors 102 (“second connectors 106”), or between two or more other connectors 102 (“third connectors 108”). Only two attachment portions 140 and two connectors 102 are labeled in FIG. 2A for ease of illustration. As discussed herein, the number, configuration, and location of the connectors 102 and attachment portions 140 may be selected to provide a desired force on one or more of the teeth when the appliance 100 is installed. According to some embodiments, the appliance 100 can comprise one or more marking regions (not shown) configured to communicate information related to the design, production, distribution, installation, and/or use of the appliance 100. The marking region(s) can comprise indicia providing and/or encoding the information of interest. The appliance 100 can include one, some, or all of the embodiments of the marking region (e.g., marking region 402, 502, 602a, 602b, 702, 802, 902a, 902b, 1002, 1102a, 1102b, 1202a, 1202b, 1302, 1402, 1502, 1602) and/or one, some, or all of the embodiments of the indicia (e.g., indicia 404, 504, 604a, 604b, 704, 804, 904a, 904b, 1004, 1104a, 1104b, 1204a, 1204b, 1304, 1404, 1504, 1604) described herein.

The attachment portions 140 may be configured to detachably couple to a securing member 160 that is bonded, adhered, or otherwise secured to a surface of one of the teeth to be moved. In some embodiments, one or more of the attachment portions 140 may be directly bonded, adhered, or otherwise secured to a corresponding tooth without a securing member or other connection interface at the tooth. The attachment portions 140 may also be referred to as “bracket connectors” or “male connector elements” herein. The different attachment portions 140 of a given appliance 100 may have the same or different shape, same or different size, and/or same or different configuration. The attachment portions 140 may comprise any of the attachment portions, bracket connectors, and/or male connector elements disclosed in U.S. patent application Ser. No. 15/370,704 (Publ. No. 2017/0156823) filed Dec. 6, 2016, U.S. patent application Ser. No. 15/929,443 (Publ. No. 2021/0007830) filed May 2, 2020, and U.S. patent application Ser. No. 15/929,444 (Publ. No. 2020/0390524) filed May 2, 2020, which are incorporated by reference herein in their entirety.

The appliance 100 may include any number of attachment portions 140 suitable for securely attaching the appliance 100 to the patient's tooth or teeth in order to achieve a desired movement. In some examples, multiple attachment portions 140 may be attached to a single tooth. The appliance 100 may include an attachment portion for every tooth, fewer attachment portions than teeth, or more attachment portions 140 than teeth. In these and other embodiments, the appliance 100 one or more of the attachment portions 140 may be configured to be coupled to one, two, three, four, five or more connectors 102.

As previously mentioned, the connectors 102 may comprise one or more first connectors 104 that extend directly between attachment portions 140. The one or more first connectors 104 may extend along a generally mesiodistal dimension when the appliance 100 is installed in the patient's mouth. In these and other embodiments, the appliance 100 may include one or more first connectors 104 that extend along a generally occlusogingival and/or buccolingual dimension when the appliance 100 is installed in the patient's mouth. In some embodiments, the appliance 100 does not include any first connectors 104.

Additionally or alternatively, the connectors 102 may comprise one or more second connectors 106 that extend between one or more attachment portions 140 and one or more connectors 102. The one or more second connectors 106 can extend along a generally occlusogingival dimension when the appliance 100 is installed in the patient's mouth. In these and other embodiments, the appliance 100 may include one or more second connectors 106 that extend along a generally mesiodistal and/or buccolingual dimension when the appliance 100 is installed in the patient's mouth. In some embodiments, the appliance 100 does not include any second connectors 106. In such embodiments, the appliance 100 would only include first connectors 104 extending between attachment portions 140. A second connector 106 and the attachment portion 140 to which it is attached may comprise an “arm,” as used herein (such as arm 130 in FIGS. 2A and 2B). In some embodiments, multiple second connectors 106 may extend from the same location along the appliance 100 to the same attachment portion 140. In such cases, the multiple second connectors 106 and the attachment portion 140 together comprise an “arm,” as used herein. The use of two or more connectors to connect two points on the appliance 100 enables application of a greater force (relative to a single connector connecting the same points) without increasing the strain on the individual connectors. Such a configuration is especially beneficial given the spatial constraints of the fixed displacement treatments herein.

Additionally or alternatively, the connectors 102 may comprise one or more third connectors 108 that extend between two or more other connectors 102. The one or more third connectors 108 may extend along a generally mesiodistal dimension when the appliance 100 is installed in the patient's mouth. In these and other embodiments, the appliance 100 may include one or more third connectors 108 that extend along a generally occlusogingival and/or buccolingual dimension when the appliance 100 is installed in the patient's mouth. In some embodiments, the appliance 100 does not include any third connectors 108. One, some, or all of the third connectors 108 may be positioned gingival to one, some, or all of the first connectors 104. In some embodiments, the appliance 100 includes a single third connector 108 that extends along at least two adjacent teeth and provides a common attachment for two or more second connectors 106. In several embodiments, the appliance 100 includes multiple non-contiguous third connectors 108, each extending along at least two adjacent teeth.

As shown in FIG. 2A, in some embodiments the appliance 100 may be configured such that all or a portion of one, some, or all of the connectors 102 are disposed proximate the patient's gingiva when the appliance 100 is installed within the patient's mouth. For example, one or more third connectors 108 may be configured such that all or a portion of the one or more third connectors 108 is positioned below the patient's gum line and adjacent to but spaced apart from the gingiva. In many cases it may be beneficial to provide a small gap (e.g., 0.5 mm or less) between the third connector(s) 108 and the patient's gingiva, as contact between the third connector(s) 108 (or any portion of the appliance 100) and the gingiva can cause irritation and patient discomfort. In some embodiments, all or a portion of the third connector(s) 108 is configured to be in direct contact with the gingiva when the appliance 100 is disposed in the patient's mouth. Additionally or alternatively, all or a portion of one or more first connectors 104 and/or second connectors 106 may be configured to be disposed proximate the gingiva.

According to some embodiments, one or more connectors 102 may extend between an attachment portion 140 or connector 102 and a joint comprising (a) two or more connectors 102, (b) two or more attachment portions 140, or (c) at least one attachment portion 140 and at least one connector 102. According to some embodiments, one or more connectors 102 may extend between a first joint comprising (a) two or more connectors 102, (b) two or more attachment portions 140, or (c) at least one attachment member and at least one connector 102, and a second joint comprising (a) two or more connectors 102, (b) two or more attachment portions 140, or (c) at least one attachment portion 140 and at least one connector 102. An example of a connector 102 extending between (a) a joint between a second and third connector 106, 108, and (b) a joint between a second connector 106 and an attachment portion 140 is depicted schematically and labeled 109 in FIG. 2B.

Each of the connectors 102 may be designed to have a desired stiffness so that an individual connector 102 or combination of connectors 102 imparts a desired force on one or more of the teeth. In many cases, the force applied by a given connector 102 may be governed by Hooke's Law, or F=k×x, where F is the restoring force exerted by the connector 102, k is the stiffness coefficient of the connector 102, and x is the displacement. In the most basic example, if a connector 102 does not exist between two points on the appliance 100, then the stiffness coefficient along that path is zero and no forces are applied. In the present case, the individual connectors 102 of the present technology may have varying non-zero stiffness coefficients. For example, one or more of the connectors 102 may be rigid (i.e., the stiffness coefficient is infinite) such that the connector 102 will not flex or bend between its two end points. In some embodiments, one or more of the connectors 102 may be “flexible” (i.e., the stiffness coefficient is non-zero and positive) such that the connector 102 can deform to impart (or absorb) a force on the associated tooth or teeth or other connector 102.

In some embodiments it may be beneficial to include one or more rigid connectors between two or more teeth. A rigid connector 102 is sometimes referred to herein as a “rigid bar” or an “anchor.” Each rigid connector 102 may have sufficient rigidity to hold and maintain its shape and resist bending. The rigidity of the connector 102 can be achieved by selecting a particular shape, width, length, thickness, and/or material. Connectors 102 configured to be relatively rigid may be employed, for example, when the tooth to be connected to the connector 102 or arm is not to be moved (or moved by a limited amount) and can be used for anchorage. Molar teeth, for example, can provide good anchorage as molar teeth have larger roots than most teeth and thus require greater forces to be moved. Moreover, anchoring one or more portions of the appliance 100 to multiple teeth is more secure than anchoring to a single tooth. As another example, a rigid connection may be desired when moving a group of teeth relative to one or more other teeth. Consider, for instance, a case in which the patient has five teeth separated from a single tooth by a gap, and the treatment plan is to close the gap. The best course of treatment is typically to move the one tooth towards the five teeth, and not vice versa. In this case, it may be beneficial to provide one or more rigid connectors between the five teeth. For all of the foregoing reasons and many others, the appliance 100 may include one or more rigid first connectors 104, one or more rigid second connectors 106, and/or one or more rigid third connectors 108.

In these and other embodiments, the appliance 100 may include one or more flexible first connectors 104, one or more flexible second connectors 106, and/or one or more flexible third connectors 108. Each flexible connector 102 may have a particular shape, width, thickness, length, material, and/or other parameters to provide a desired degree of flexibility. According to some embodiments of the present technology, the stiffness of a given connector 102 may be tuned via incorporation of a one or more resiliently flexible biasing portions 150. As shown schematically in FIG. 2B, one, some, or all of the connectors 102 may include one or more biasing portion 150, such as springs, each configured to apply a customized force specific to the tooth to which it is attached.

As depicted in the schematic shown in FIG. 2C, the biasing portion(s) 150 may extend along all or a portion of the longitudinal axis L1 of the respective connector 102 (only the longitudinal axis L1 for second connector 106 and the longitudinal axis L2 for third connector 108 is labeled in FIG. 2C). The direction and magnitude of the force and torque applied on a tooth by a biasing portion 150 depends, at least in part, on the shape, width, thickness, length, material, shape set conditions, and other parameters of the biasing portion 150. As such, one or more aspects of the biasing portion 150 (including the aforementioned parameters) may be varied so that the corresponding arm 130, connector 102, and/or biasing portion 150 produces a desired tooth movement when the appliance 100 is installed in the patient's mouth. Each arm 130 and/or biasing portion 150 may be designed to move one or more teeth in one, two, or all three translational directions (i.e., mesiodistal, buccolingual, and occlusogingival) and/or in one, two, or all three rotational directions (i.e., buccolingual root torque, mesiodistal angulation and mesial out-in rotation).

The biasing portions 150 of the present technology can have any length, width, shape, and/or size sufficient to move the respective tooth towards a desired position. In some embodiments, one, some, or all of the connectors 102 may have one or more inflection points along a respective biasing portion 150. The connectors 102 and/or biasing portions 150 may have a serpentine configuration such that the connector 102 and/or biasing portion 150 doubles back on itself at least one or more times before extending towards the attachment portion 140. For example, in some embodiments the second connectors 106 double back on themselves two times along the biasing portion 150, thereby forming first and second concave regions facing in generally different directions relative to one another. The open loops or overlapping portions of the connector 102 corresponding to the biasing portion 150 may be disposed on either side of a plane P (FIG. 2C) bisecting an overall width W (FIG. 2C) of the arm 130 and/or connector 102 such that the extra length of the arm 130 and/or connector 102 is accommodated by the space medial and/or distal to the arm 130 and/or connector 102. This allows the arm 130 and/or connector 102 to have a longer length (as compared to a linear arm) to accommodate greater tooth movement, despite the limited space in the occlusal-gingival or vertical dimension between any associated third connector 108 and the location at which the arm 130 attaches to the tooth.

It will be appreciated that the biasing portion 150 may have other shapes or configurations. For example, in some embodiments the connector 102 and/or biasing portion 150 may include one or more linear regions that zig-zag towards the attachment portion 140. One, some, or all of the connectors 102 and/or biasing portions 150 may have only linear segments or regions, or may have a combination of curved and linear regions. In some embodiments, one, some, or all of the connectors 102 and/or biasing portions 150 do not include any curved portions.

According to some examples, a single connector 102 may have multiple biasing portions 150 in series along the longitudinal axis of the respective connector 102. In some embodiments, multiple connectors 102 may extend between two points along the same or different paths. In such embodiments, the different connectors 102 may have the same stiffness or different stiffnesses.

In those embodiments where the appliance 100 has two or more connectors 102 with biasing portions 150, some, none, or all of the connectors 102 may have the same or different lengths, the same or different widths, the same or different thicknesses, the same or different shapes, and/or may be made of the same or different materials, amongst other properties. In some embodiments, less than all of the connectors 102 have biasing portions 150. Connectors 102 without biasing portions 150 may, for example, comprise one or more rigid connections between a rigid third connector 108 and the attachment portion 140. In some embodiments, none of the connectors 102 of the appliance 100 have a biasing portion 150.

According to some embodiments, for example as depicted schematically in FIG. 2A, the appliance 100 may include a single, continuous, substantially rigid third connector (referred to as “anchor 120”) and a plurality of flexible arms 130 extending away from the anchor 120. When the appliance 100 is installed in the patient's mouth, each of the arms 130 may connect to a different one of the teeth to be moved and exerts a specific force on its respective tooth, thereby allowing an operator to move each tooth independently. Such a configuration provides a notable improvement over traditional braces in which all of the teeth are connected by a single archwire, such that movement of one tooth can cause unintentional movement of one or more nearby teeth. The independent and customized tooth movement enabled by the appliances of the present technology allows the operator to move the teeth from an original tooth arrangement (“OTA”) to a final tooth arrangement (“FTA”) more efficiently, thereby obviating periodic adjustments, reducing the number of office visits, and reducing or eliminating patient discomfort, and reducing the overall treatment time (i.e., the length of time the appliance is installed in the patient's mouth) by at least 50% relative to the overall treatment time for traditional braces.

The anchor 120 may comprise any structure of any shape and size configured to comfortably fit within the patient's mouth and provide a common support for one or more of the arms 130. In many embodiments, the anchor 120 is disposed proximate the patient's gingiva when the appliance 100 is installed within the patient's mouth, for example as shown in FIG. 2B. For instance, the appliance may be designed such that, when installed in the patient's mouth, all or a portion of the anchor 120 is positioned below the patient's gum line and adjacent but spaced apart from the gingiva. In many cases it may be beneficial to provide a small gap (e.g., 0.5 mm or less) between the anchor 120 (or any portion of the appliance 100) and the patient's gingiva as contact between the anchor 120 and the gingiva can cause irritation and patient discomfort. In some embodiments, all or a portion of the anchor 120 is configured to be in contact with the gingiva when the appliance 100 is disposed in the patient's mouth.

The anchor 120 may be significantly more rigid than the arms 130 such that the equal and opposite forces experienced by each of the arms 130 when exerting a force on its respective tooth are countered by the rigidity of the anchor 120 and the forces applied by the other arms 130, and do not meaningfully affect the forces on other teeth. As such, the anchor 120 effectively isolates the forces experienced by each arm 130 from the rest of the arms 130, thereby enabling independent tooth movement.

According to some embodiments, for example as shown schematically in FIGS. 2A and 2B, the anchor 120 comprises an elongated member having a longitudinal axis L2 (see FIG. 2C) and forming an arched shape configured to extend along a patient's jaw when the appliance 100 is installed. In these and other embodiments, the anchor 120 may be shaped and sized to span two or more of the patient's teeth when positioned in the patient's mouth. In some examples, the anchor 120 includes a rigid, linear bar, or may comprise a structure having both linear and curved segments. In these and other embodiments, the anchor 120 may extend laterally across all or a portion of the patient's mouth (e.g., across all or a portion of the palate, across all or a portion of the lower jaw, etc.) and/or in a generally anterior-posterior direction. Moreover, the appliance 100 may comprise a single anchor or multiple anchors. For example, the appliance 100 may comprise multiple, discrete, spaced apart anchors, each having two or more arms 130 extending therefrom. In these and other embodiments, the appliance 100 may include one or more other connectors extending between adjacent arms 130.

Any and all of the features discussed above with respect to anchor 120 applies to any of the third connectors 108 disclosed herein.

As shown in FIG. 2B, each of the arms 130 may extend between a proximal or first end portion 130a and a distal or second end portion 130b, and may have a longitudinal axis L extending between the first end portion 130a and the second end portion 130b. The first end portion 130a of one, some, or all of the arms 130 may be disposed at the anchor 120. In some embodiments, one, some, or all of the arms 130 are integral with the anchor 120 such that the first end portion 130a of such arms are continuous with the anchor 120. The arms 130 may extend from the anchor 120 at spaced intervals along the longitudinal axis L2 of the, as shown in FIG. 2A. In some embodiments, the arms 130 may be spaced at even intervals relative to each other, or at uneven intervals relative to each other, along the longitudinal axis L2 of the anchor 120.

One, some, or all of the arms 130 may include an attachment portion 140 at or near the second end portion 130b. In some embodiments, for example as shown in FIGS. 2A-2C, one or more of the arms 130 is cantilevered from the anchor 120 such that the second end portion 130b of the cantilevered arm(s) 130 has a free distal end portion 130b. In these and other embodiments, a distal terminus of the attachment portion 140 may coincide with a distal terminus of the arm 130. The attachment portion 140 may be configured to detachably couple the respective arm 130 to a securing member (e.g., a bracket) that is bonded, adhered, or otherwise secured to a surface of one of the teeth to be moved. In some embodiments, the attachment portion 140 may be directly bonded, adhered, or otherwise secured to a corresponding tooth without a securing member or other connection interface at the tooth.

Referring to still to FIGS. 2A and 2B, one, some, or all of the arms 130 may include one or more resiliently flexible biasing portions 150, such as springs, each configured to apply a customized force, torque or combination of force and torque specific to the tooth to which it is attached. The biasing portion(s) 150 may extend along all or a portion of the longitudinal axis L1 of the respective arm 130 between the anchor 120 and the attachment portion 140. The direction and magnitude of the force and torque applied on a tooth by a biasing portion 150 depends, at least in part, on the shape, width, thickness, length, material, shape set conditions, and other parameters of the biasing portion 150. As such, one or more aspects of the arm 130 and/or biasing portion 150 (including the aforementioned parameters) may be varied so that the arm 130 and/or biasing portion 150 produce a desired tooth movement when the appliance 100 is installed in the patient's mouth. Each arm 130 and/or biasing portion 150 may be designed to move one or more teeth in one, two, or all three translational directions (i.e., mesiodistal, buccolingual, and occlusogingival) and/or in one, two, or all three rotational directions (i.e., buccolingual root torque, mesiodistal angulation and mesial out-in rotation).

The biasing portions 150 of the present technology can have any length, width, shape, and/or size sufficient to move the respective tooth towards a desired FTA. In some embodiments, one, some, or all of the arms 130 may have one or more inflection points along a respective biasing portion 150. The arms 130 and/or biasing portions 150 may have a serpentine configuration such that the arm 130 and/or biasing portion 150 doubles back on itself at least one or more times before extending towards the attachment portion 140. In FIG. 2B, the arm 130 doubles back on itself two times along the biasing portion 150, thereby forming first and second concave regions facing in generally different directions relative to one another. The open loops or overlapping portions of the arm 130 corresponding to the biasing portion 150 may be disposed on either side of a plane P bisecting an overall width W of the arm 130 such that the extra length of the arm 130 is accommodated by the space medial and/or distal to the arm 130. This allows the arm 130 to have a longer length (as compared to a linear arm) to accommodate greater tooth movement, despite the limited space in the occlusal-gingival or vertical dimension between the anchor 20 and the location at which the arm 130 attaches to the tooth.

It will be appreciated that the biasing portion 150 may have other shapes or configurations. For example, in some embodiments the arm 130 and/or biasing portion 150 may include one or more linear regions that zig-zag towards the attachment portion 140. One, some, or all of the arms 130 and/or biasing portions 150 may have only linear segments or regions, or may have a combination of curved and linear regions. In some embodiments, one, some, or all of the arms 130 and/or biasing portions 150 do not include any curved portions.

According to some examples, a single arm 130 may have multiple biasing portions 150. The multiple biasing portions 150 may be in series along the longitudinal axis L1 of the respective arm 120. In some embodiments, multiple arms 130 may extend in parallel between two points along the same path or along different paths. In such embodiments, the different arms 130 may have the same stiffness or different stiffnesses.

In those embodiments where the appliance 100 has two or more arms 130 with biasing portions 150, some, none, or all of the arms 130 may have the same or different lengths, the same or different widths, the same or different thicknesses, the same or different shapes, and/or may be made of the same or different materials, amongst other properties. In some embodiments, less than all of the arms 130 have biasing portions 150. Arms 130 without biasing portions 150 may, for example, comprise one or more rigid connections between the anchor 120 and the attachment portion 140. In some embodiments, none of the arms 130 of the appliance 100 have a biasing portion 150.

The appliances of the present technology may include any number of arms 130 suitable for repositioning the patient's teeth while taking into account the patient's comfort. Unless explicitly limited to a certain number of arms in the specification, the appliances of the present technology may comprise a single arm, two arms, three arms, five arms, ten arms, sixteen arms, etc. In some examples, one, some, or all of the arms 130 of the appliance may be configured to individually connect to more than one tooth (i.e., a single arm 130 may be configured to couple to two teeth at the same time). In these and other embodiments, the appliance 100 may include two or more arms 130 configured to connect to the same tooth at the same time.

Any portion of the appliances of the present technology may include a biasing portion 150. For example, in some embodiments, portions thereof (e.g., the anchor(s), the arm(s), the biasing portion(s), the attachment portion(s), the link(s), etc.) may comprise one or more superelastic materials.

Additional details related to the individual directional force(s) applied via the biasing portion 150 or, more generally the arm 130, are described in U.S. application Ser. No. 15/370,704, now U.S. Pat. No. 10,383,707, issued Aug. 20, 2019, the disclosure of which is incorporated by reference herein in its entirety.

The appliances disclosed herein and/or any portion thereof (e.g., the anchor(s), the arm(s), the biasing portion(s), the attachment portion(s), the link(s), etc.) may comprise one or more superelastic materials. The appliances disclosed herein and/or any portion thereof (e.g., the anchor(s), the arm(s), the biasing portion(s), the attachment portion(s), the link(s), etc.) may comprise Nitinol, stainless steel, beta-titanium, cobalt chrome, MP35N, 35N LT, one or more metal alloys, one or more polymers, one or more ceramics, and/or combinations thereof.

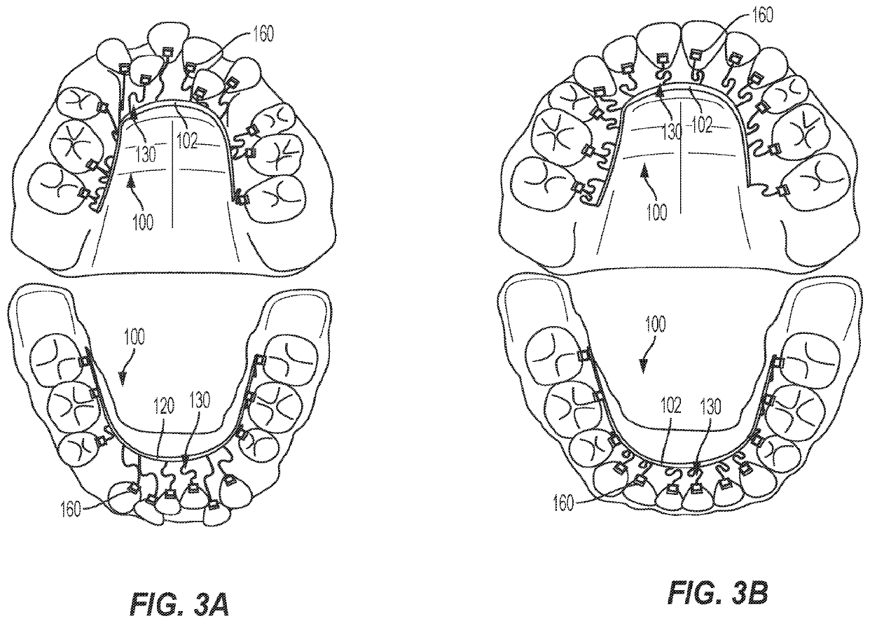

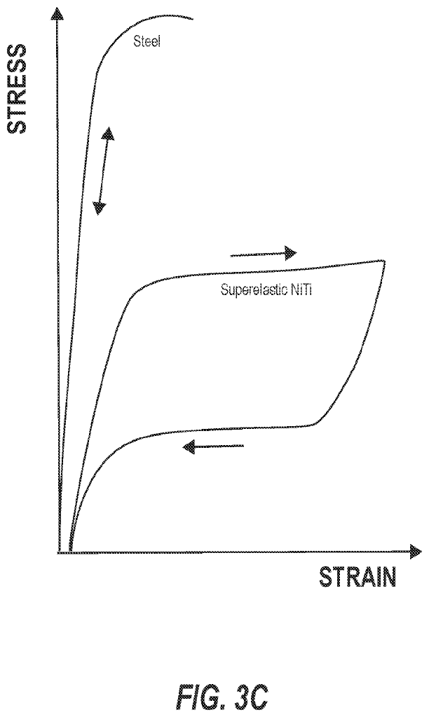

FIGS. 3A and 3B are elevation views of the appliance 100 installed on both the upper and lower arches of a patient's mouth with the arms 130 coupled to securing members 160 attached to the lingual surfaces of the teeth. It will be appreciated that the appliance 100 of one or both of the upper and lower arches may be positioned proximate a buccal side of a patient's teeth, and that the securing members 160 and/or arms 130 may alternatively be coupled to the buccal surface of the teeth.

As described herein, in some embodiments the appliance 100 includes a marking region (not shown) configured to communicate information corresponding to an intended position and/or orientation of the appliance 100 with respect to the patient's teeth. For example, the marking region can comprise indicia communicating which arch (e.g., upper or lower) the appliance should be secured to, whether the appliance 100 should be positioned at the lingual or buccal side of the patient's teeth, which tooth or teeth a specific arm 130 should be secured to, etc. In some embodiments, the marking region is configured to associate the appliance 100 with a specific patient. Such information regarding the patient and the intended position and/or orientation of the appliance 100 reduces installation time, which benefits both the practitioner and the patient.

FIG. 3A shows the teeth in an OTA with the arms 130 in a deformed or loaded state, and FIG. 3B shows the teeth in the FTA with the arms 130 in a substantially unloaded state. When the arms 130 are initially secured to the securing members 160 when the teeth are in the OTA, the arms 130 are forced to take a shape or path different than their “as designed” configurations. Because of the inherent memory of the resilient biasing portions 150, the arms 130 impart a continuous, corrective force on the teeth to move the teeth towards the FTA, which is where the biasing portions 150 are in their as-designed or unloaded configurations. As such, tooth repositioning using the appliances of the present technology can be accomplished in a single step, using a single appliance. In addition to enabling fewer office visits and a shorter treatment time, the appliances of the present technology greatly reduce or eliminate the pain experienced by the patient as the result of the teeth moving as compared to braces. With traditional braces, every time the orthodontist makes an adjustment (such as installing a new archwire, bending the existing archwire, repositioning a bracket, etc.), the affected teeth experience a high force which is very painful for the patient. Over time, the applied force weakens until eventually a new wire is required. The appliances of the present technology, however, apply a movement-generating force on the teeth continuously while the appliance is installed, which allows the teeth to move at a slower rate that is much less painful (if painful at all) for the patient. Even though the appliances disclosed herein apply a lower and less painful force to the teeth, because the forces being applied are continuous and the teeth can move independently (and thus more efficiently), the appliances of the present technology arrive at the FTA faster than traditional braces or aligners, as both alternatives require intermediate adjustments.

In many embodiments, the movement-generating force is lower than that applied by traditional braces. In those embodiments in which the appliance comprises a superelastic material (such as nitinol), the superelastic material behaves like a constant force spring for certain ranges of strain, and thus the force applied does not drop appreciably as the tooth moves. For example, as shown in the stress-strain curves of nitinol and steel in FIG. 3C, the curve for nitinol is relatively flat compared to that of steel. Thus, the superelastic connectors, biasing portions, and/or arms of the present technology apply essentially the same stress for many different levels of strain (e.g., deflection). As a result, the force applied to a given tooth stays constant as the teeth move during treatment, at least up until the teeth are very close or in the final arrangement. The appliances of the present technology are configured to apply a force just below the pain threshold, such that the appliance applies the maximum non-painful force to the tooth (or teeth) at all times during tooth movement. This results in the most efficient (i.e., fastest) tooth movement without pain.

In some embodiments, tooth repositioning may involve multiple steps performed progressively, by using multiple appliances. In such embodiments, each appliance can comprise a marking region configured to communicate information corresponding to an intended order of use of the appliance. For example, the marking region can be configured to communicate that the appliance is the first appliance in a set of three appliances. Embodiments involving multiple steps (or multiple appliances, or both) may include one or more intermediate tooth arrangements (ITAs) between an original tooth arrangement (OTA) and a desired final tooth arrangement (FTA). Likewise, the appliances disclosed herein may be designed to be installed after a first or subsequently used appliance had moved the teeth from an OTA to an ITA (or from one ITA to another ITA) and was subsequently removed. Thus, the appliances of the present technology may be designed to move the teeth from an ITA to an FTA (or to another ITA). Additionally or alternatively, the appliances may be designed to move the teeth from an OTA to an ITA, or from an OTA to an FTA without changing appliances at an ITA.

In some embodiments, the appliances disclosed herein may be configured such that, once installed on the patient's teeth, the appliance cannot be removed by the patient. In some embodiments, the appliance may be removable by the patient.

Any of the example appliances or appliance portions described herein may be made of any suitable material or materials, such as, but not limited to Nitinol (NiTi), stainless steel, beta-titanium, cobalt chrome or other metal alloy, polymers, or ceramics, and may be made as a single, unitarily-formed structure or, alternatively, in multiple separately-formed components connected together in single structure. However, in particular examples, the rigid bars, bracket connectors and loop or curved features of an appliance (or portion of an appliance) described in those examples are made by cutting a two dimensional (2D) form of the appliance from a 2D sheet of material and bending the 2D form into a desired 3D shape of the appliance, according to processes as described in U.S. patent application Ser. No. 15/370,704 (Publ. No. 2017/0156823), filed Dec. 6, 2016, U.S. patent application Ser. No. 15/929,442 (Publ. No. 2020/0345455), filed May 2, 2020, or other suitable processes.

### III. Methods of Manufacturing

The particular processes described herein are exemplary only and may be modified as appropriate to achieve the desired outcome (e.g., the desired force applied to each tooth by the appliance, the desired material properties of the appliance, etc.). In various embodiments, other suitable methods or techniques can be utilized to fabricate an orthodontic appliance. Moreover, although various aspects of the methods disclosed herein refer to sequences of steps, in various embodiments the steps can be performed in different orders, two or more steps can be combined together, certain steps may be omitted, and additional steps not expressly discussed can be included in the process as desired.

As noted above, in some embodiments an orthodontic appliance is configured to be coupled to a patient's teeth while the teeth are in an OTA. In this position, elements of the appliance exert customized loads on individual teeth to urge them toward a desired FTA. For example, an arm 130 of the appliance 100 can be coupled to a tooth and configured to apply a force so as to urge the tooth in a desired direction toward the FTA. In one example, an arm 130 of the appliance 100 can be configured to apply a tensile force that urges the tooth lingually along the facial-lingual axis. By selecting the appropriate dimensions, shape, shape set, material properties, and other aspects of the arms 130, a customized load can be applied to each tooth to move each tooth from its OTA toward its FTA. In some embodiments, the arms 130 are each configured such that little or no force is applied once the tooth to which the arm 130 is coupled has achieves its FTA. In other words, the appliance 100 can be configured such that the arms 130 are at rest in the FTA state.

The method may begin with obtaining data (e.g., position data) characterizing the patient's OTA. In some embodiments the operator may obtain a digital representation of the patient's OTA, for example using optical scanning, cone beam computed tomography (CBCT), scanning of patient impressions, or other suitable imaging technique to obtain position data of the patient's teeth, gingiva, and optionally other adjacent anatomical structures while the patient's teeth are in the original or pre-treatment condition.

The method may further comprise obtaining data (e.g., position data) characterizing the patient's intended or desired FTA, and in many cases generating a digital representation of the patient's FTA. The data characterizing the FTA can include coordinates (e.g., X,Y,Z coordinates) for each of the patient's teeth and the gingiva. Additionally or alternatively, such data can include positioning of each of the patient's teeth relative to other ones of the patient's teeth and/or the gingiva.

In some embodiments, segmentation software (e.g., iROK Digital Dentistry Studio) be used to create individual virtual teeth and gingiva from the OTA data. Suitable software can be used to move the virtual teeth to their FTA positions. In some cases digital models of securing members can be added to the OTA digital model (e.g., by an operator selecting positions on the tooth surface for placement of securing members thereon). Suitable software can be used to move the virtual teeth with the attached securing members from the OTA to a desired final position.

In some embodiments a heat treatment fixture digital model can be obtained. In some embodiments, the heat treatment fixture digital model can correspond to and/or be derived from the OTA digital model and/or the FTA digital model. For example, the OTA digital model and/or the FTA digital model can be modified (e.g., using MeshMixer or other suitable modeling software) in a variety of ways to render a model suitable for manufacturing a heat treatment fixture. In some embodiments, the OTA or FTA digital model can be modified to replace the securing members (which are configured to couple to arms 130 of an appliance 100 (FIG. 2A)) with members (which can be configured to facilitate temporary coupling of the heat treatment fixture to the appliance for shape-setting). Additionally or alternatively, the OTA or FTA digital model can be modified to enlarge or thicken the gingiva, to remove one or more of the teeth, and/or to add structural components for increased rigidity. In some embodiments, enlarging or thickening the gingiva may be done to ensure portions (e.g., the anchor) of the fabricated appliance, which is based in part on the OTA digital model or the FTA digital model, does not engage or contact the patient's gingiva when the appliance is installed. As a result, modifying the OTA or FTA digital model as described herein may be done to provide a less painful teeth repositioning experience for the patient.

The method may further comprise obtaining an appliance digital model. As used herein, the term “digital model” and “model” are intended to refer to a virtual representation of an object or collection of objects. For example, the term “appliance digital model” refers to the virtual representation of the structure and geometry of the appliance, including its individual components (e.g., the anchor, arms, biasing portions, attachment portions, etc.). In some embodiments, a substantially planar digital model of the appliance is generated based at least in part on the heat treatment fixture digital model (and/or the OTA digital model and/or the FTA digital model). According to some examples, a contoured or 3D appliance digital model generally corresponding to the FTA can first be generated that conforms to the surface and attachment features of the heat treatment fixture digital model. In some embodiments, the 3D appliance digital model can include generic arm portions and securing members, without particular geometries, dimensions, or other properties of the arms being selected or defined by a particular patient. The 3D appliance digital model may then be flattened to generate a substantially planar appliance digital model. In some embodiments, the particular configuration of the arms 130 (e.g., the geometry of biasing portions 150, the position along the anchor 120 (FIG. 2A), etc.) can then be selected so as to apply the desired force to urge the corresponding tooth (to which the arm 130 is attached) from its OTA toward its FTA. As noted previously, in some embodiments the arms are configured so as to be substantially at rest or in a substantially unstressed state when at the FTA. The selected arm configurations can then be substituted or otherwise incorporated into the planar appliance digital model. In some embodiments, the planar appliance digital model and/or the 3D appliance digital model includes a digital representation of the indicia to be included in the marking region of the appliance.

In some cases, it may be beneficial to evaluate an intended appliance design prior to fabricating a physical appliance based on the intended appliance design to assess how the physical appliance would perform during treatment. For example, because the pre-installation form of the appliance is based at least in part on a desired FTA, the position of one or more portions of the appliance may shift relative to the gingiva once the physical appliance is installed in the patient's mouth (e.g., with the patient's teeth in the OTA). As a result, one or more shifted positions of the physical appliance may cause pain for the patient that may reduce treatment compliance and/or satisfaction.

In some embodiments, finite element analysis (or other suitable computational techniques) can be used to manipulate the 3D appliance digital model to assess its performance prior to fabrication. For example, the 3D appliance digital model can be virtually deformed (e.g., using finite element analysis) into a position for engagement with the patient's teeth in the OTA. The resulting virtual model represents the appliance digital model after it has been deformed into position to be engaged with the patient's teeth in the OTA. An output of the virtual deformation can be evaluated to assess whether the physical appliance will function as intended. Based on the evaluation of the output, the intended appliance design can be modified as needed, or a final appliance design can be obtained. For example, evaluation of the output of the virtual deformation can reveal that a portion of the appliance digital model impinges on the gingiva digital model. As a result, the design of the appliance may be modified, and the evaluation may be repeated until the appliance digital model no longer impinges on the gingiva. This process may be repeated iteratively until a satisfactory appliance design is achieved.

Next, the heat treatment fixture can be fabricated. For example, using the heat treatment fixture digital model, the heat treatment fixture can be cast, molded, 3D printed, or otherwise fabricated using suitable materials configured to withstand heating for shape setting of an appliance thereon.

In some embodiments, fabricating the appliance includes first fabricating the appliance in a planar configuration based on the planar appliance digital model. For example, a pattern of the planar form of the final device can be cut out of a sheet of material to get a planar member. In some embodiments, the appliance is cut out of a sheet of Nitinol or other metal using laser cutting, water jet, stamping, or other suitable technique. The thickness of the material can be varied across the appliance, for example by electropolishing, etching, depositing, or otherwise manipulating the material of the appliance to achieve the desired material properties. In some embodiments, the sheet of material can be modified to include the marking region and/or the indicia prior to the planar appliance being cut from the sheet of material. In some embodiments, the sheet of material can be modified to include the marking region and/or the indicia while the planar appliance is being cut from the sheet of material. For example, a single machine can cut the appliance and the indicia at the same time. In some embodiments, the marking region and/or the indicia can be added to the appliance after the planar appliance has been cut from the sheet of material.

According to some embodiments, the planar member (e.g., as 3D-printed or as cut out from a sheet of material) can be bent or otherwise manipulated into the desired arrangement (e.g., substantially corresponding to the FTA) to form a 3D appliance for treatment. In some embodiments, the planar member can be bent into position by coupling the planar member to a heat treatment fixture. The heat treatment fixture may be, for example, the physical form of the previously-obtained heat treatment fixture digital model. For example, the arms of the planar member can be removably coupled to hook members of the heat treatment fixture, and optionally ligature wire or other temporary fasteners can be used to secure the arms or other portions of the appliance to the heat treatment fixture. The resulting assembly (i.e., the appliance fastened to the heat treatment fixture) can then be heated to shape-set the appliance into its final form, which can correspond or substantially correspond to the FTA. As a result, the appliance is configured to be in an unstressed state in the FTA. The shape set appliance can then be removed from the heat treatment fixture. In some embodiments, the marking region and/or the indicia can be added to the appliance after the appliance has been shape set.

In operation, the appliance can then be installed in the patient's mouth (e.g., by bending or otherwise manipulating arms of the appliance to be coupled to brackets of the patient's teeth while in the OTA). Due to the shape set of the appliance and the geometry of the arms and anchor, the arms will tend to urge each tooth away from its OTA and toward the FTA.

### IV. Orthodontic Device Marking Regions

As described herein, an orthodontic device (e.g., an orthodontic appliance, an indirect bonding tray, a device for manufacturing an orthodontic appliance, etc.) can include a marking region that communicates information related to the design, production, distribution, installation, and/or use of the device. The marking region, for example, can include indicia configured to communicate, present, and/or encode information corresponding to an origin of the device, an identity of the patient, an instruction for installing the device, and/or other information related to one of the above-noted processes. As used herein, a “marking region” or a “marker” comprises a portion of the device that communicates information. The marking region can include indicia to provide such information. The indicia can comprise a recessed portion of the device, an opening extending through an entire thickness of the device, a raised or protruding portion of the device, and/or material that has been added to the device. In various embodiments, the indicia include, for example, letters, numbers, symbols, colors, shapes, patterns, and/or other markings that directly provide and/or encode the information of interest. As used herein, “indicia” may refer to: a single marking; a group of markings; a plurality of single markings, each communicating a different piece of information; and/or a plurality of groups of markings, each communicating a different piece of information. The indicia can be human-readable such that a human operator can interpret the information provided by the indicia without the aid of a machine for decryption. Additionally or alternatively, the indicia can be machine-readable.

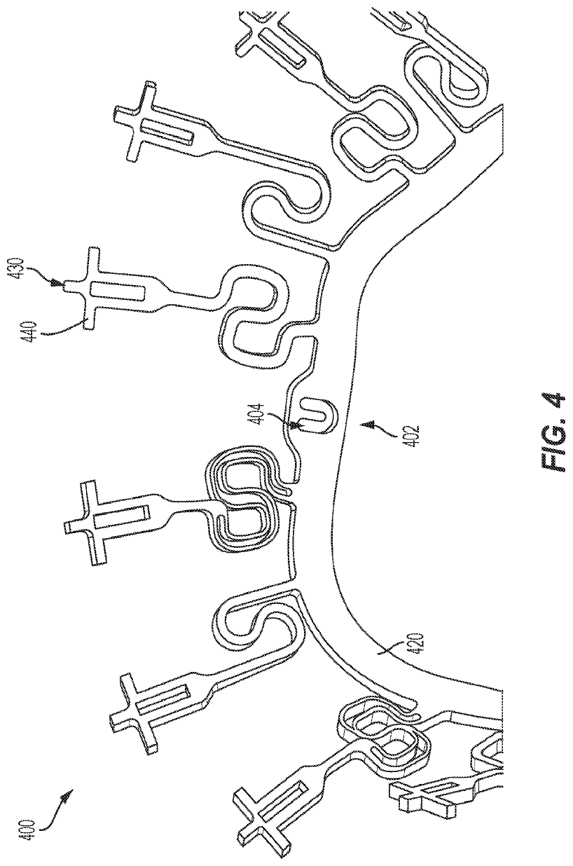

FIGS. 4-14 depict various embodiments of appliances 400, 500, 600, 700, 800, 900, 1000, 1100, 1200, 1300, and 1400, with one or more marking regions 402, 502, 602, 702, 802, 902, 1002, 1102, 1202, 1302, and 1402. Any of the appliances 400, 500, 600, 700, 800, 900, 1000, 1100, 1200, 1300, and 1400 can be similar to any of the embodiments of another of the appliances 100, 400, 500, 600, 700, 800, 900, 1000, 1100, 1200, 1300, and 1400 disclosed herein. For example, arms 430 of appliance 400 can be similar to arms 130 of appliance 100, anchor 620 of appliance 600 can be similar to anchor 420 of appliance 400, etc.

FIG. 4 depicts an orthodontic appliance 400 with a marking region 402 configured in accordance with several embodiments of the present technology. The appliance 400 can include an anchor 420, a plurality of arms 430, and a plurality of attachment portions 440. As previously mentioned, the marking region 402 can be a portion of the appliance that is configured to communicate useful information. For example, the marking region 402 can be a portion of the appliance 400 that includes indicia 404 providing such information. The indicia 404 can include at least one of a number, a letter, a symbol, a color, a shape, or a pattern. In some embodiments, the indicia 404 can include a letter indicating which of the patient's teeth and/or jaws the appliance 400 is configured to be secured to. For example, as shown in FIG. 4, the indicia 404 can include the letter “U” to indicate that the appliance 400 should be secured to the teeth in the patient's upper dental arch. Moreover, as described in greater detail herein, the indicia 404 can comprise an opening extending completely (see FIG. 4) or partially through a thickness of the appliance 400 (e.g., along a buccolingual dimension).

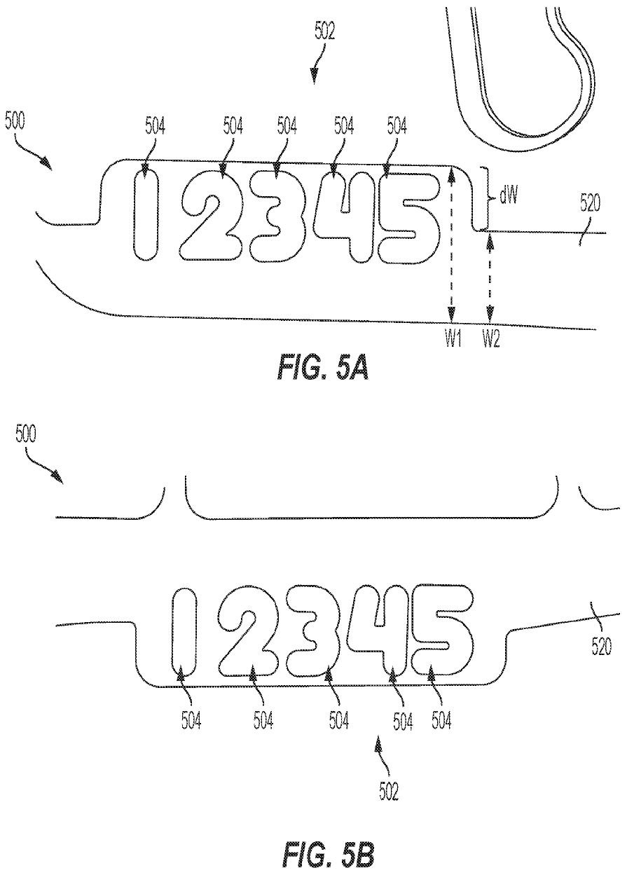

According to some embodiments, for example as shown in FIG. 4, the marking region 402 is positioned at, near, and/or along the anchor 420. Additionally or alternatively, the marking region 402 can be positioned at one or more of the arms 430. In any case, the portion of the appliance 400 comprising the marking region 402 can have an occlusogingival width that that is greater than the portion(s) of the appliance 400 immediately adjacent to the marking region 402 to increase the space available on the anchor 420 for the indicia 404. As used herein, an occlusogingival width of the appliance 400 refers to a dimension of the appliance along a generally occlusogingival dimension when the appliance 400 is positioned within the patient's mouth. The occlusogingival width of the marking region 402 can be greater than the occlusogingival width of the surrounding and/or adjacent portions of the appliance 400 in an occlusal direction (see, for example, FIGS. 4 and 5A), in a gingival direction (see, for example, FIG. 5B), or in both an occlusal direction and a gingival direction. In some embodiments, a thickness of the appliance 400 at the marking region may be greater or less than a thickness of the rest of the appliance 400. As used herein, a thickness of the appliance 400 refers to a dimension of the appliance along a generally buccolingual dimension when the appliance 400 is positioned within the patient's mouth.

The location of the marking region 402 and/or indicia 404 along the appliance 400 can provide useful information a handler of the appliance 400. For example, the marking region 402 and/or indicia 404 can be positioned at or adjacent to a midline of the appliance 400 (see FIG. 4) to indicate the intended installation orientation of the appliance 400. In some embodiments, the marking region 402 can be distal of the midline of the appliance 400 (not shown in FIG. 4). The shape and/or configuration of the indicia 404 itself may communicate information related to the information provided by the location of the indicia 404, and in some embodiments the shape and/or configuration of the indicia 404 itself may communicate different information than the information provided by the location of the indicia 404. An example of the latter scenario is depicted in FIG. 4 in which the ‘U’ opening in the anchor 420 communicates to an operator that the appliance 400 is to be installed on the patient's upper jaw, while the location of the marking region 402 (at the midline) communicates a general intended orientation of the appliance 400 relative to the patient's teeth. In some embodiments, the shape and/or configuration of the indicia 404 itself does not communicate any information and instead the useful indicator is the location of the indicia 404 on the appliance 400. For example, a simple tick mark can be positioned at or near the midline of the appliance 400 to communicate the general intended orientation of the appliance 400 when installed in the patient's mouth.

Although FIG. 4 depicts a single marking region 402, the appliance 400 may comprise multiple marking regions 402. The marking regions 402 can be positioned adjacent to one another or spaced apart from one another. The marking regions 402 can be positioned at the anchor 420, the arms 430, or both the anchor 420 and the arms 430. In some embodiments, the marking regions 402 are each configured to communicate a different type of information (e.g., a first marking region 402 associates the appliance 400 with a specific patient, a second marking region 402 indicates an intended position of the appliance 400 with respect to the patient's teeth, etc.). In some embodiments, a single marking region 402 is configured to communicate multiple types of information (e.g., a serial number of the appliance, a patient identifier, etc.).

Although FIG. 4 depicts indicia 404 comprising a single element (in this case, a single letter), the marking region 402 may include indicia 404 comprising multiple letters, numbers, symbols, colors, shapes, patterns, and/or combinations thereof. For example, as shown in FIGS. 5A and 5B, an appliance 500 can comprise an anchor 520 with a marking region 502 comprising indicia 504 including multiple numbers. The indicia 504 can directly provide and/or encode an identity of an associated patient, a serial number of the appliance 500, an intended position of the appliance 500 with respect to the patient's teeth, etc. In some embodiments, the indicia 504 comprises a unique combination of letters, numbers, symbols, colors, shapes, and/or patterns for each piece of information provided by the indicia 504. For example, the indicia 504 may include a combination of letters, numbers, symbols, colors, shapes, and/or patterns that is unique to the specific patient to associate the appliance 500 with a specific patient. In some embodiments, the indicia 504 can include a combination of letters, numbers, symbols, colors, shapes, and/or patterns to provide and/or encode a serial number that is unique to the appliance 500.

As previously noted, the marking region 502 can communicate a single type of information or multiple types of information. In some embodiments, the marking region 502 includes multiple types of indicia 504 to communicate multiple types of information. For example, the marking region 502 can include first indicia providing information that associates the appliance with a specific patient, second indicia providing information corresponding to an intended position and/or orientation of the appliance with respect to the patient's teeth, third indicia providing information corresponding to an origin of the appliance, etc. In some embodiments, each type of indicia 504 includes a single type of symbol (e.g., first indicia includes one or more numbers, second indicia includes one or more letters, etc.). In some embodiments, each type of indicia 504 can include multiple types of symbols (e.g., each of first indicia and second indicia includes numbers and letters, etc.). Each type of indicia 504 can include unique types of symbols or each type of indicia 504 can include the same types of symbols. Additionally or alternatively, an arrangement of individual symbols of the indicia 504 can be unique for each type of indicia 504. For example, indicia 504 corresponding to a serial number of the appliance 500 can comprise a combination of letters followed by a combination of numbers, indicia 504 associating the appliance 500 with a specific patient can comprise a combination of numbers alone, etc.

As shown in FIG. 5A, the anchor 520 can have a first occlusogingival width W1 at the marking region 502 that is different than a second occlusogingival width W2 of the anchor 520 at one or more locations adjacent to the marking region 502 by a difference dW. As used herein, an occlusogingival width (e.g., the first occlusogingival width W1, the second occlusogingival width W2, etc.) of the anchor 520 refers to a dimension of the anchor 520 along a generally occlusogingival dimension when the appliance 500 is positioned within the patient's mouth. The first occlusogingival width W1 at the marking region 502 can be greater than the second occlusogingival width W2 to increase the space available on the anchor 520 for the indicia 504. The first occlusogingival width W1 can be greater than the second occlusogingival width W2 in an occlusal direction (see FIG. 5A), in a gingival direction (see FIG. 5B), or in both an occlusal direction and a gingival direction.

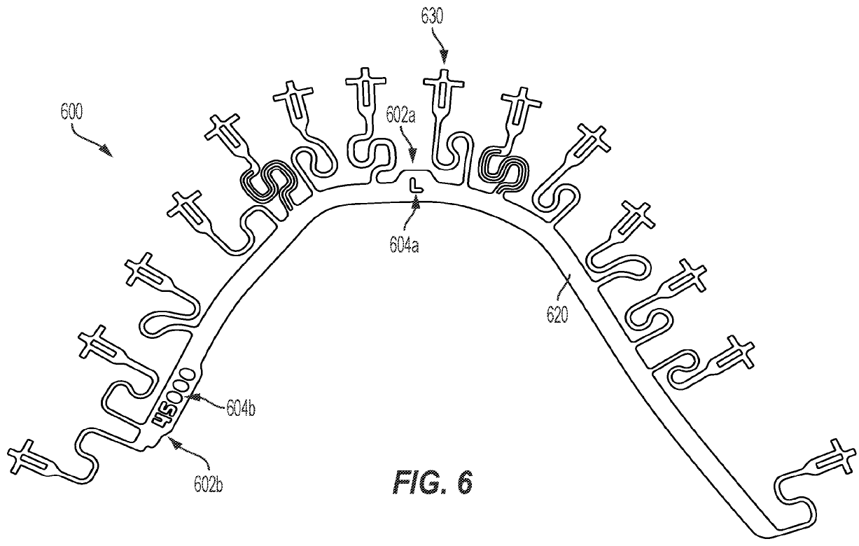

Although FIGS. 4-5B depict appliances 400, 500 each comprising a single marking region 402, 502, an appliance of the present technology can have more than one marking region. For example, FIG. 6 depicts an appliance 600 having a first marking region 602a and a second marking region 602b (collectively “marking regions 602”). Although two marking regions 602 are depicted in FIG. 6, various numbers of marking regions 602 are possible. For example, the appliance 600 can have zero marking regions, one marking region 602, two marking regions 602, three marking regions 602, four marking regions 602, or more. The marking regions 602 can be spaced apart from one another (see FIG. 6) or positioned adjacent to one another (not shown). The marking regions 602 can be positioned at the anchor 620, at arms 630 of the appliance 600, both the anchor 620 and the arms 630, a connector, etc. of the appliance 600. For example, as shown in FIG. 6, both marking regions 602 can be positioned at the anchor 620 of the appliance 600.

As described herein, the marking region(s) 602 can be configured to communicate information regarding the appliance 600, a patient, etc. Additionally or alternatively, the marking region(s) 602 can include indicia 604 providing the information. For example, the first marking region 602a can include first indicia 604a providing first information and the second marking region 602b can include second indicia 604b providing second information. In some embodiments, the first indicia 604a includes symbols and/or a combination of symbols differing from the second indicia 604b such that the first information provided by the first indicia 604a is different than the second information provided by the second indicia 604b. For example, in FIG. 6 the first indicia 604a includes a letter 1′ and the second indicia 604b includes a series of numbers. The ‘L’ first indicia 604a can communicate to an operator that the appliance 600 is configured to be installed on a patient's lower jaw and/or the location of the marking region 602 (at the midline) can communicate a general intended orientation of the appliance 600 relative to the patient's teeth. The numerical second indicia 604b can associate the appliance 600 with a specific patient, provide information corresponding to an origin of the appliance 600, etc. In some embodiments, the marking regions 602 can be configured to communicate the same information. Each marking region 602 can be configured to communicate one type of information (e.g., just a serial number of the appliance) or multiple types of information (e.g., a serial number of the appliance and a patient identifier, etc.).

As shown in FIG. 6, one or both of the marking regions 602 can have an occlusogingival width that is different than an occlusogingival width of the anchor 620 at one or more locations adjacent to the marking region(s) 602. The occlusogingival width of the marking regions 602 can be greater than the occlusogingival width of the anchor 620 (see FIG. 6) or the occlusogingival width of the marking region(s) 602 can be less than an occlusogingival width of the anchor 620. For example, as shown in FIG. 6, the first marking region 602a can have an occlusogingival width that is greater in an occlusal direction with respect to an occlusogingival width of the anchor 620 at one or more locations adjacent to the first marking region 602a and/or the second marking region 602b can have an occlusogingival width that is greater in a gingival direction with respect to an occlusogingival width of the anchor 620 at one or more locations adjacent to the second marking region 602b. As previously described, the occlusogingival width of the marking region(s) 602 can be greater than the occlusogingival width of the anchor 620 in an occlusal direction and/or in a gingival direction. In some embodiments, the occlusogingival width of the marking regions 602 is the same as or similar to the occlusogingival width of the anchor 620.

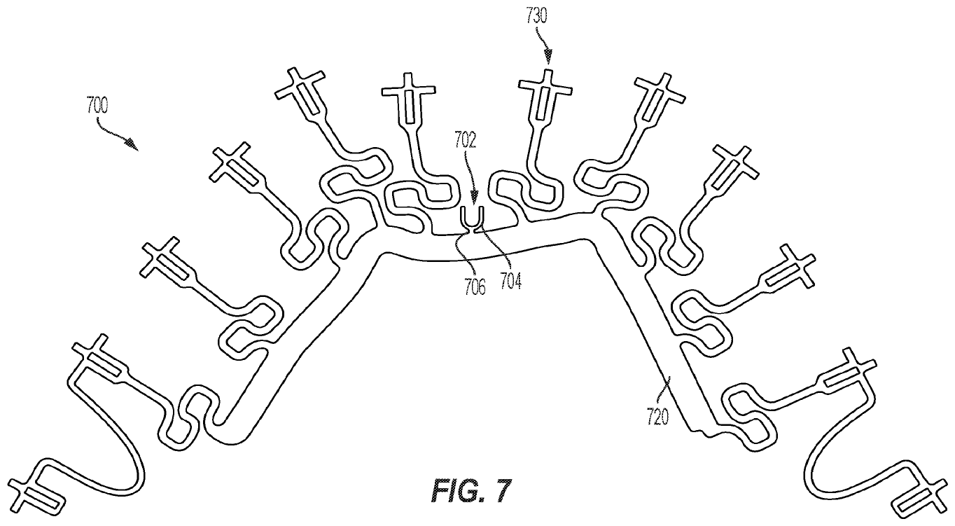

In some embodiments, for example as shown in FIG. 7, an appliance 700 includes a marking region 702 positioned occlusally of an anchor 720 of the appliance 700. Additionally or alternatively, the marking region 702 can be positioned gingivally, distally, mesially, lingually, or facially of the anchor 720 (not shown). The marking region 702 can include indicia 704 providing information, as described herein. The marking region 702 and/or indicia 704 can be continuous with the anchor 720, one or more arms 730 of the appliance 700, and/or or another portion of the appliance 700 and have a shape that directly corresponds to the information to be communicated. For example, the indicia 704 shown in FIG. 7 is a portion of the appliance 700 in the shape of the letter ‘U’ and indicates that that the appliance 700 is configured to be installed on a patient's upper jaw. In some embodiments, the marking region 702 and/or indicia 704 are cut from the same sheet of material as some or all of the rest of the appliance 700.

As shown in FIG. 7, in some embodiments the indicia 704 is spaced apart from the rest of the appliance 700 by a neck portion 706. As such, the neck portion 706 connects the indicia 704 to the anchor 720, arm 730, and/or other portion of the appliance 700. The neck portion 706 can have a mesiodistal width measured along a mesiodistal dimension that is less than a mesiodistal width of the of the indicia 704 and/or a mesiodistal width of the anchor 720. Additionally or alternatively, the neck portion 706 can have an occlusogingival width that is less than, the same as, or more than an occlusogingival width of the of the indicia 704. In some embodiments, the neck portion 706 can optionally be configured to facilitate visualization of the indicia 704. For example, the neck portion 706 can separate the indicia 704 from the rest of the appliance 700 by a sufficient amount for an operator to visually distinguish the indicia 704 from the rest of the appliance and/or interpret the indicia 704.

In various embodiments, the neck portion 706 is structurally weakened such that the marking region 702 can be separated from the appliance 700 before, during, or after installation of the appliance 700. For example, a width (e.g., a mesiodistal width, an occlusogingival width, etc.) and/or a thickness of the neck portion 706 can be sufficiently small to enable an operator to easily separate the marking region 702 from the appliance 700 at the neck portion 706 (e.g., by hand, with clippers, etc.). In some embodiments, a thickness of the marking region 702 is substantially equivalent to a thickness of the anchor 720 and/or a thickness of one or more of the arms 730. In some embodiments, the thickness of the marking region 702 is different than the thickness of the anchor 720 and/or the thickness of one or more of the arms 730. A shape and/or size of the marking region 702, including the indicia 704 and/or the neck portion 706, can be configured to ensure that the marking region 702 does not contact the arms 730 or anchor 720 of the appliance 700 during installation and/or use of the appliance 700. In some embodiments the indicia 704 extends directly from the anchor 720 (e.g., the marking region 702 does not include a neck portion 706).

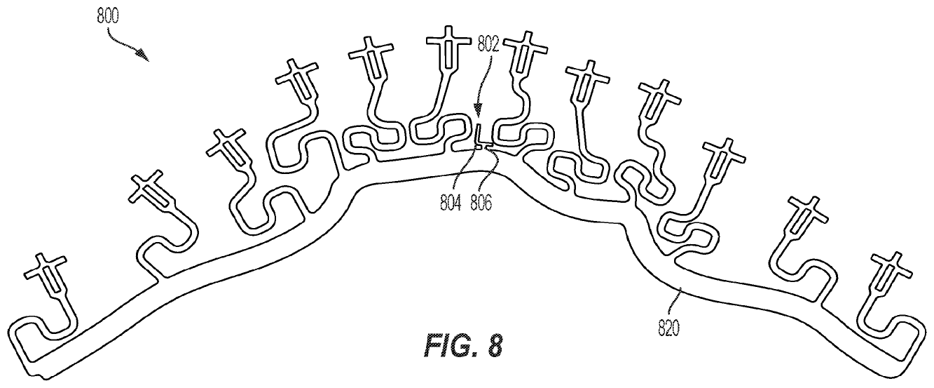

The appliance 800 shown in FIG. 8 can be similar to any of the appliances disclosed herein, except as described below. FIG. 8 shows an appliance 800 with a marking region 802 positioned occlusally of an anchor 820 and including ‘L’ indicia 804. The ‘L’ indicia can indicate, for example, that that the appliance 800 is configured to be installed on a patient's lower jaw. As previously described with respect to FIG. 7, the marking region 802 can include a neck portion 806 connecting the indicia 804 to the appliance 800. In some embodiments, the marking region 802 does not include a neck portion 806.

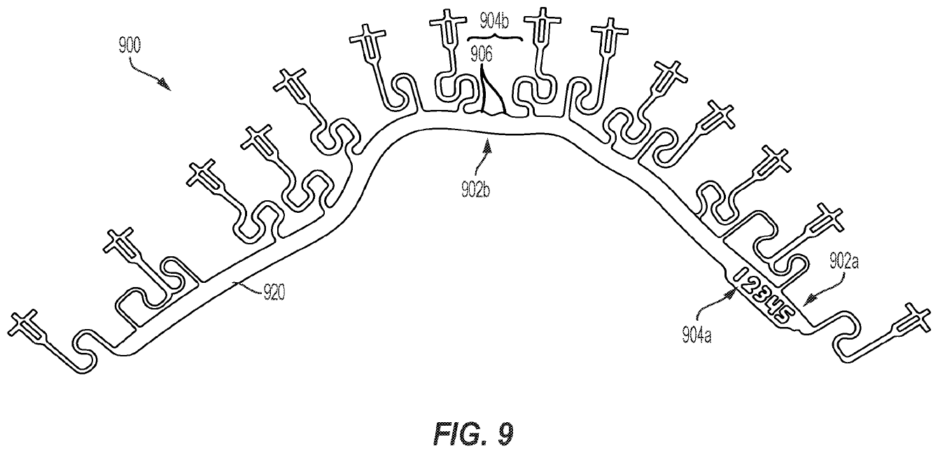

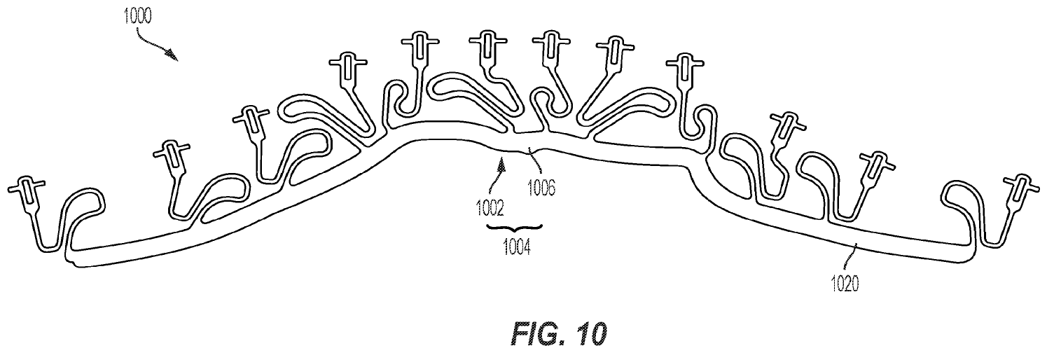

In some embodiments, a marking region can comprise a uniquely shaped portion of the appliance that provides information regarding an origin of the appliance, an identity of the patient, an instruction for installing the appliance, and/or other information as described herein. FIG. 9 depicts an example of such a marking region. FIG. 9 shows an appliance 900 including an anchor 920 having a first marking region 902a and a second marking region 902b. The first marking region 902a includes first indicia 904a and the second marking region 902b includes second indicia 904b. The first indicia 904a can comprise a series of numbers, and the second indicia 904b can comprise two protuberances 906 adjacent one another along an occlusal side of the anchor 920. Thus, the second marking region 902b is identifiable by an operator in that it comprises a portion of the anchor 920 where one side of the anchor 920 has an uncharacteristic undulating shape. In FIG. 9, both of the protuberances 906 extend occlusally from the anchor 920. The protuberances 906 can extend occlusally, gingivally, distally, mesially, buccally, and/or lingually with respect to the anchor 920 and/or another portion of the appliance 900. In some embodiments, the protuberances 906 extend in the same direction. In some embodiments, the protuberances 906 do not extend in the same direction. The number, shape, and/or location of the protuberances 906 can provide information about the appliance 900, the patient, etc. For example, two protuberances 906 (see FIG. 9) can communicate that the appliance 900 is configured to be installed on a patient's upper jaw. Additionally or alternatively, the protuberances 906 can be positioned at or near the midline of the appliance 900 to communicate an intended orientation of the appliance 900 with respect to the patient's teeth. Still, additional configurations are possible. For example, FIG. 10 depicts an appliance 1000 comprising a marking region 1002 having indicia 1004 comprising a protuberance 1006. The appliance 1000 can be similar to the appliance 900 of FIG. 9 (or any other appliance disclosed herein) except as described below. As shown in FIG. 10, the indicia 1004 can comprise a single protuberance 1006 along a gingival side of the anchor 1020. The single protuberance 1006 can communicate that the appliance 1000 is configured to be installed on a patient's lower jaw. As shown in FIGS. 9 and 10, a protuberance (e.g., protuberances 906, protuberance 1006, etc.) can have a generally triangular shape. Additionally or alternatively, a protuberance can have any other suitable shape such as, but not limited to, rectangular, circular, etc.

As previously discussed, and as shown in FIG. 9, the first marking region 902a can be spaced apart from the second marking region 902b. In some embodiments, the first marking region 902a is positioned distal of a midline of the anchor 920 and/or the second marking regions 902b is positioned at or adjacent to a midline of the anchor 920. The first and second marking regions 902a, 902b can have different shapes (see FIG. 9), similar shapes (not shown), or the same shape (not shown). For example, the first marking region 902a is shown with a generally rectangular shape in FIG. 9. However, the first marking region 902a could also have an irregular and/or identifiable shape similar to the second marking region 902b.

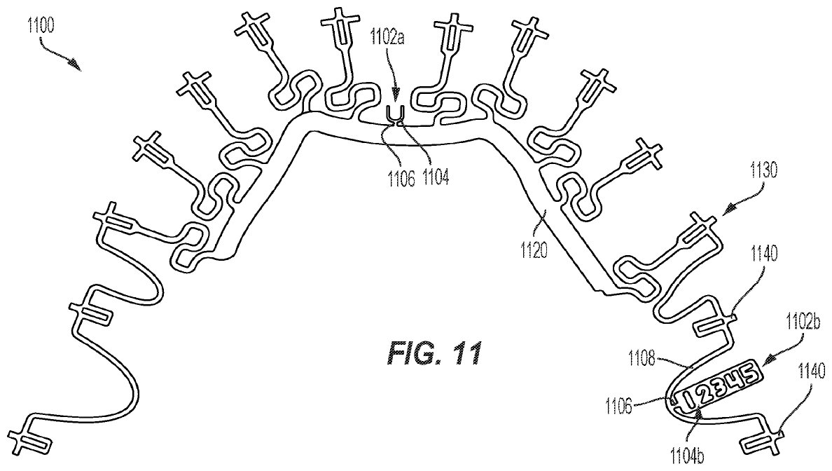

In some embodiments, for example as shown in FIG. 11, an appliance 1100 can comprise a first marking region 1102a including first indicia 1104a and a second marking region 1102b including second indicia 1104b. The first marking region 1102a can have a shape substantially corresponding to the shape of the first indicia 1104a (e.g., the first marking region 1102a can have a ‘U’ shape corresponding to the ‘U’ shaped indicia 1104a). Additionally or alternatively, the second marking region 1102b can have a generally rectangular shape with the second indicia 1104b positioned within the generally rectangular perimeter of the second marking region 1102b. The second indicia 1104b can comprise a marking on a surface of the second marking region 1102b, an opening or recess in the second marking region 1102b, a raised portion of the second marking region 1102b, etc. Although FIG. 11 depicts the second indicia 1104b comprising a single line of numbers extending occlusally from the anchor 1120, any of the indicia disclosed herein can comprise multiple rows and/or multiple columns of letters, numbers, symbols, shapes, patterns, etc. The rows or columns can extend in one or more directions (e.g., occlusally, gingivally, mesially, distally, lingually, buccally, etc.). As previously described with reference to FIG. 7, the first marking region 1102a and/or the second marking region 1102b can include a neck portion 1106 connecting the marking region to the appliance 1100. The first marking region 1102a can be connected to the anchor 1120 of the appliance 1100 (see FIG. 11), to an arm 1130 of the appliance 1100, and/or another suitable location as disclosed herein. The second marking region 1102b can be connected to a connector 1108 extending between attachment portions 1140 of the appliance 1100 (see FIG. 11), the anchor 1120, one or more arms 1130, etc. The neck portion 1106 can be configured to facilitate visualization of the indicia 1104 and/or the neck portion 1106 can be configured to permit the marking regions 1102 to be separated from the appliance 1100 before, during, or after installation of the appliance 1100. A shape and/or size of the marking regions 1102, including the indicia 1104 and/or the neck portions 1106, can be configured to ensure that the marking regions 1102 do not unintentionally contact other portions of the appliance 1100 during installation and/or use of the appliance 1100.

As shown in FIG. 11, the first marking region 1102a and/or the second marking region 1102b can be positioned gingivally and/or occlusally of an anchor 1120, an arm 1130, and/or a connector 1108 of the appliance 1100. Additionally or alternatively, the first marking region 1102a and/or the second marking region 1102b can be positioned lingually or buccally of the anchor 1120. The first marking region 1102a and/or the second marking region 1102b can be substantially planar as shown in FIG. 11 and/or can comprise one or more bends such that the marking region(s) 1102 extend in two or more directions.

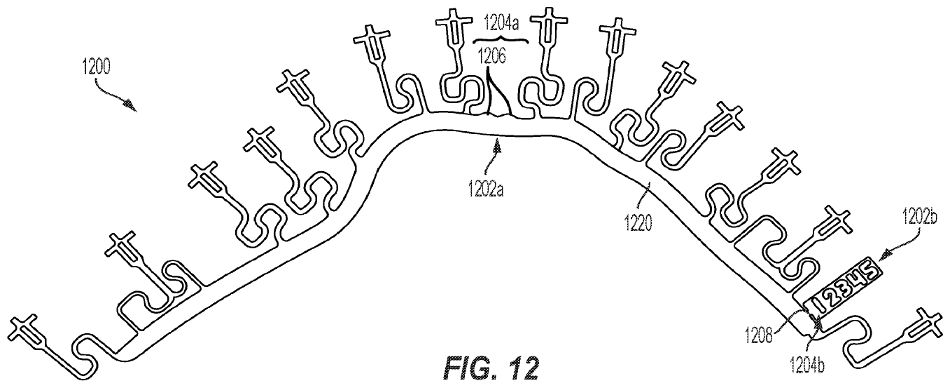

In some embodiments, for example as shown in FIG. 12, an appliance 1200 can comprise a first marking region 1202a including first indicia 1204a and a second marking region 1202b including second indicia 1204b. The first indicia 1204a can comprise two protuberances 1206 adjacent one another along an occlusal side of an anchor 1220 of the appliance 1200, and the second indicia 1204b comprise a series of numbers. As previously described, the protuberances 1206 can extend occlusally, gingivally, distally, mesially, buccally, and/or lingually with respect to the anchor 1220 and/or another portion of the appliance 1200. In some embodiments, the protuberances 1206 extend in the same direction. In some embodiments, the protuberances 1206 do not extend in the same direction. The number, shape, and/or location of the protuberances 1206 can provide information about the appliance 1200, the patient, etc. For example, two protuberances 1206 (see FIG. 12) can communicate that the appliance 1200 is configured to be installed on a patient's upper jaw. Additionally or alternatively, the protuberances 1206 can be positioned at or near the midline of the appliance 1200 to communicate an intended orientation of the appliance 1200 with respect to the patient's teeth. As previously described, the second marking region 1202b can have a generally rectangular shape with the second indicia 1204b positioned within the generally rectangular perimeter of the second marking region 1202b. The second marking region 1202b can include a neck portion 1208 connecting the marking region to the appliance 1200 (e.g., at an arm of the appliance 1200 (not shown), at the anchor 1202 of the appliance 1200 (see FIG. 12), etc.).

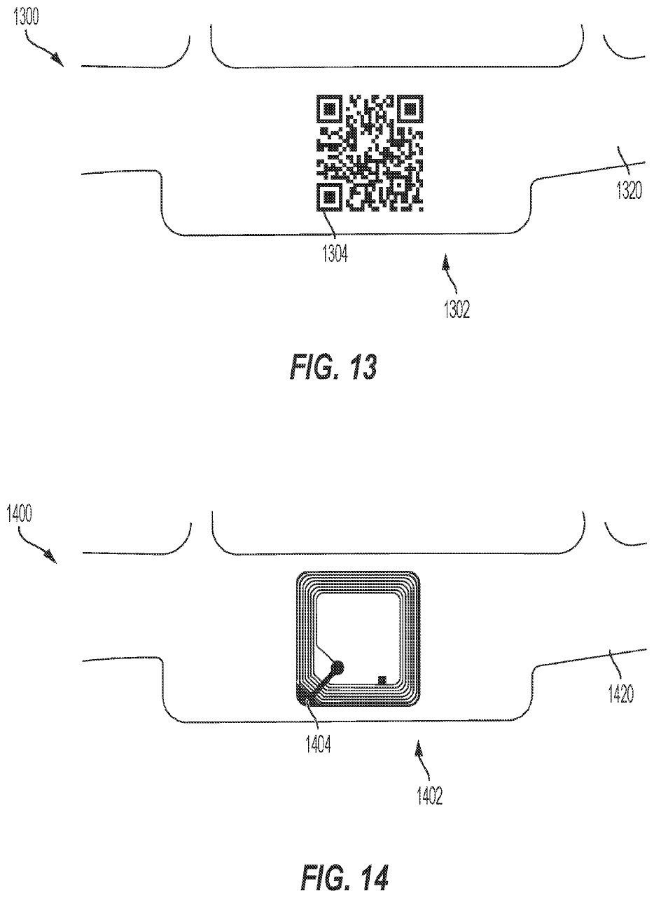

FIG. 13 depicts a portion of an appliance 1300 having a marking region 1302 at an anchor 1320. In some embodiments, the marking region 1302 comprises machine-readable indicia 1304. For example, as shown in FIG. 13, the indicia 1304 can comprise Quick Read (QR) code that contains information regarding the appliance 1300, the patient, installation of the appliance 1300, etc. Additionally or alternatively, the indicia 1304 can comprise iQR code, micro QR code, FrameQR code, a barcode, and/or other suitable machine-readable code. In some embodiments, the indicia 1304 is configured to be read by a machine (e.g., a computer, a smartphone, etc.) via a camera. The indicia 1304 may be configured to be interpreted by the machine to determine the information communicated by the indicia 1304. In some embodiments, the indicia 1304 is configured to be displayed by the machine (e.g., via a user interface). The information can be used by the machine and/or communicated to an operator or another machine to facilitate the design, production, distribution, installation, and/or use of the appliance 1300. For example, the information can be communicated from the machine to an operator to associate the appliance 1300 with a specific patient to ensure that the operator installs the appliance 1300 in the mouth of the correct patient.

FIG. 14 depicts a portion of an appliance 1400 having a marking region 1402 at an anchor 1420. In some embodiments, the marking region 1402 can comprise machine-readable indicia 1404 comprising a radio-frequency identification (RFID) tag. The RFID tag can be configured to transmit digital data to an RFID reader to communicate information regarding the appliance 1400, the patient, installation of the appliance 1400, etc. The RFID tag can include an antenna, a microchip, and a substrate, and the RFID tag can be passive, active, or battery-assisted passive.

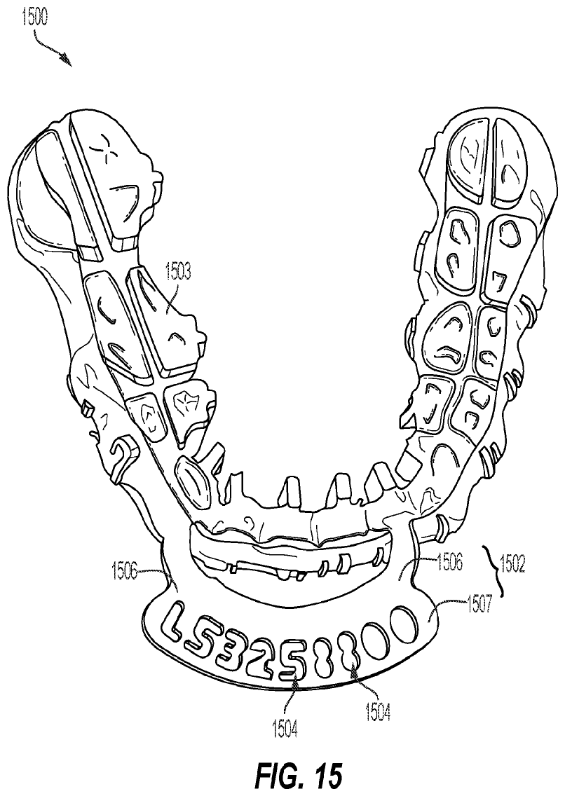

A marking region in accordance with several embodiments of the present technology can be configured to provide useful information with regards to a variety of orthodontic devices. Such orthodontic devices may include, but are not limited to, orthodontic appliances configured to move a patient's teeth, retainers configured to maintain a position of a patient's teeth, indirect bonding trays configured to position orthodontic brackets with respect to a patient's teeth, devices for manufacturing an orthodontic device, and others. For example, FIG. 15 depicts an orthodontic device 1500 comprising a body portion 1503 configured to be releasably secured to a patient's teeth and a marking region 1502 having indicia 1504 configured to communicate information about the device 1500, the patient, use of the device 1500, etc. In some embodiments, the device 1500 comprises an indirect bonding tray or a direct bonding tray. The device 1500 can be configured to position an orthodontic bracket and/or an orthodontic appliance (such as any of the appliances disclosed herein) at or adjacent to the patient's teeth in a desired position so that the orthodontic bracket and/or appliance can be secured to the patient's teeth. The marking region 1502 can be integral with the body portion 1503 (e.g., formed of the same material and/or one continuous body) or may be a separate component that is fixedly or detachably coupled to the body portion 1503. In some embodiments, the marking region 1502 comprises a first portion 1507 that includes the indicia 1504 and one or more second portions 1506 extending between the first portion 1507 and the body portion 1503. In some embodiments, the marking region 1502 is positioned at the body portion 1503 of the device 1500. In such embodiments and others, the indicia 1504 can comprise a marking on the body portion 1503, a recess or an opening in the body portion 1503, a raised portion of the body portion 1503, etc. In various embodiments, the device 1500 can include one or more marking regions include indicia indicating a tooth that a corresponding region of the body portion of the device should be secured to.

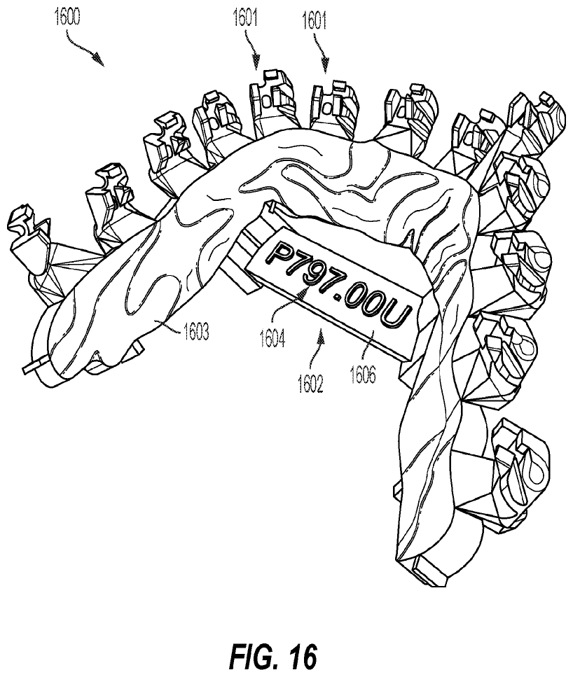

Marking regions of the present technology can also provide useful information regarding devices configured for manufacturing orthodontic appliances or devices. For example, FIG. 16 depicts a device 1600 for forming a shape of an orthodontic appliance during manufacturing thereof. The device 1600 can be the same as or similar to the heat treatment fixtures disclosed herein. The device 1600 can comprise a body portion 1603 and one or more securing portions 1601 (only a few labeled) extending away from the body portion 1603 and configured to secure another device or appliance to the device 1600. In some embodiments, for example as shown in FIG. 16, the device 1600 optionally includes a stabilizer 1606 extending between different portions of the device 1600. In FIG. 16, the stabilizer 1606 comprises a substantially rigid member extending across the arch of the body portion 1603. In these and other embodiments, the device 1600 can include more than one stabilizer and/or the stabilizer may extend between different portions of the device 1600.

As shown in FIG. 16, the device 1600 can include a marking region 1602 having indicia 1604 providing information about the device 1600, the corresponding appliance, the patient, etc. In some embodiments the marking region 1602 can be positioned at the stabilizer 1606. Additionally or alternatively, the marking region 1602 can be positioned at the body portion 1603 and/or at one or more of the securing portions 1601. In some embodiments, the marking region 1602 is positioned away from the other portions of the device 1600 (e.g., the body portion, the securing portions, etc.) and is connected to one or more of the other portions by a neck portion or other connector. As shown in FIG. 16, the indicia 1604 can comprise a raised portion of the marking region 1602 and/or the indicia 1604 can comprise a series of numbers, letters, symbols, etc. In some embodiments, the marking region 1602 and/or indicia 1604 can indicate a desired positioning of the appliance (or precursor to the appliance) on the device 1600.

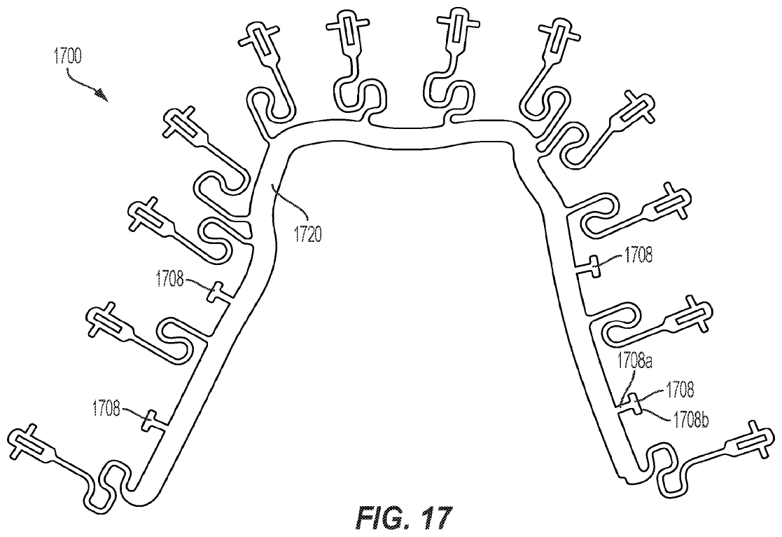

Some appliances of the present technology can include one or more features for securing the appliance to an elastic member and/or a permanent or temporary anchorage device (TAD) to facilitate intrusion and/or extrusion of one or more of the patient's teeth. For example, the orthodontic appliance 1700 depicted in FIG. 17 comprises a plurality of projection(s) 1708 configured to be directly or indirectly secured to an elastic member and/or an anchorage device. In some embodiments, the projection(s) 1708 are configured to be secured to one end of an elastic band, and the other end of the elastic band is configured to be secured to an anchorage device or another appliance or portion thereof. In some embodiments the projection(s) 1708 are configured to be secured directly to the anchorage device. The anchorage device is configured to be secured through the gingiva to a patient's palate (or other adjacent hard or soft tissue) and provide a substantially fixed anchor point around which the teeth can be moved. Examples of anchorage devices include a plate, a screw, a bar, a platform, a ring and/or other structures. Anchorage devices can be especially useful for treating molar intrusions, for example. In certain examples, the projections 1708 can be configured to be coupled to any of the TADs disclosed in U.S. application Ser. No. 16/865,323, filed May 2, 2020.

The projection(s) 1708 can extend occlusally away from the anchor 1720, gingivally, lingually, buccally, mesially, and/or distally. The appliance 1700 may include a single projection 1708 or a plurality of projections 1708. In those embodiments comprising a plurality of projections 1708, one, some, or all of the individual projections 1708 can extend in the same or different directions and can have the same or different shapes. The projection(s) 1708 can be substantially planar as shown in FIG. 17 and/or one or more of the projections 1708 can be bent, folded, twisted, or otherwise non-planar. In some embodiments, the projection(s) 1708 are substantially T-shaped. In such embodiments, the projection(s) 1708 can have a first portion 1708a extending away from the anchor 1720 (for example, in an occlusal or gingival direction) and a second portion 1708b positioned at a distal end region of the first portion 1708a and angled with respect to the first portion 1708a. According to some methods of use, a looped end of an elastic band can be positioned over the first portion 1708a and under the second portion 1708b such that, at least under tension, the second portion 1708b prevents the elastic band from slipping off the projection 1708. Additionally or alternatively, the projection(s) 1708 can have any suitable shape for being secured to an elastic or another orthodontic component.

In some embodiments, the appliance 1700 includes one or more marking regions and/or indicia in addition to the one or more projections 1708. In some embodiments, for example as shown in FIG. 17, the appliance 1700 does not include any marking regions and/or indicia.

Before or after the orthodontic appliance 1700 is secured to one or more securing elements (which may be already secured to the patient's teeth or not yet secured to the patient's teeth), a clinician may secure one or more of the projections 1708 of the appliance 1700 to one or more anchorage devices, then secure the anchorage device(s) to the patient's palate. In some methods of use, before or after the orthodontic appliance 1700 is secured to the securing elements (which may be already secured to the patient's teeth or not yet secured to the patient's teeth), a clinician may secure the anchorage device(s) to the patient's palate, then secure one, some, or all of the projections 1708 of the appliance 1700 to one or more anchorage devices. In particular examples, the inclusion, number, type, and/or location of the projections 1708 may depend, at least in part, on the type of malocclusion to be addressed and other factors specific to each patient.

A marking region of the present technology (e.g., marking region 402, 502, 602a, 602b, 702, 802, 902a, 902b, 1002, 1102a, 1102b, 1202a, 1202b, 1302, 1402, 1502, 1602) can include any indicia configured to communicate information related to the design, production, distribution, installation, and/or use of the device. In some embodiments, the indicia are configured for use with an automatic identification and data capture (AIDC) technology. Such indicia may include a magnetic stripe or an integrated circuit card. The indicia can include sensors such as low-energy Bluetooth beacons to communicate information to a machine and/or an operator.

Any of the indicia (e.g., indicia 404, 504, 604a, 604b, 704, 804, 904a, 904b, 1004, 1104a, 1104b, 1204a, 1204b, 1304, 1404, 1504, 1604) described herein can be formed by modifying the device at any stage in the manufacturing processes described herein. For example, as described herein, the appliance can be modified to include the indicia prior to cutting the appliance from a sheet of material in a substantially planar configuration, while cutting the appliance from a sheet of material in a substantially planar configuration, after cutting the planar appliance and prior to shape setting the appliance into a 3D configuration, after shape setting the appliance, etc. According to several embodiments, the appliance and the indicia can be cut at the same time and/or with the same machine. Devices such as device 1500 and device 1600 can be modified to include indicia prior to manufacturing the devices (e.g., a virtual design of the devices can be modified), while forming the devices, and/or after forming the devices.

For any of the uses described herein, the indicia can comprise one or more recesses extending partially through a thickness of the device and/or one or more openings extending entirely through a thickness of the device. As used herein, “thickness” refers to a dimension of the device along a generally buccolingual dimension when the device is positioned within the patient's mouth. The recesses or openings can be created in the appliance via any suitable process such as, but not limited to, laser cutting, engraving, milling, wire electrical discharge machining, water jetting, etching, punching, or stamping. In various embodiments, the indicia comprise one or more raised portions (e.g., protrusions, ridges, ribs, etc.) of the device that extend in a buccal direction and/or a lingual direction to a greater extent than adjacent portions of the device. The raised portions can be created by embossing, milling, or machining the device. For example, the raised portions can be created by milling a predetermined thickness of material from a surface of the appliance at locations adjacent and/or away from the intended indicia such that, when the milling process is complete, the portions of the device comprising the indicia are raised with respect to the surrounding portions of the device. According to several embodiments, the indicia comprise one or more materials that have been added to a surface of the device. For example, the indicia can comprise an ink, a film, and/or a coating added to the device. Materials can be added to a surface of the device via suitable processes such as vacuum deposition, additive manufacturing, electroplating, printing, coating, etc. Additionally or alternatively, the indicia can comprise a surface treatment of the device and/or a material property of the device. In various embodiments, for example in embodiments in which the indicia comprise a QR code or an RFID tag, the indicia are added to the device via adhesive bonding, welding, soldering, or another suitable joining process.

The marking region and/or indicia can be positioned at a lingual-facing side of the device and/or a facial-facing side of the device. In some embodiments, the indicia are visible to an operator upon visual inspection of the device. The indicia can be visible to the operator before the device is installed, during installation of the device, and/or after the device is installed. Visualization of the indicia can be facilitated by (or require) an external detection aid, such as a light source (for example, an ultraviolet light source). In some embodiments, the indica are luminescent or fluorescent. In some embodiments, machine-readable indicia may not be visible to an operator during at least one of designing, manufacturing, distributing, installing, or using the device. For example, an RFID tag does not require a direct line of sight with the corresponding RFID reader to be readable by an RFID reader. Accordingly, such indicia do not need to be positioned on a surface of the device and may be embedded within the device or otherwise blocked from visualization by an operator. In some embodiments, machine-readable indicia may be configured to be visible to an operator at all times.

In some embodiments, any of the indicia disclosed herein are permanent and cannot be nondestructively separated from the device. In such embodiments, the indicia remain associated with the appliance for the duration of manufacturing, distribution, installation, and use of the device. However, in some cases the indicia may not be required to maintain its utility throughout each of these processes. For example, the utility of indicia communicating an instruction for installing the appliance may be substantially reduced once the device has been installed in the patient's mouth. Accordingly, in some embodiments the indicia are temporary and can be separated from the device without compromising the structural integrity and/or clinical utility of the device. In some embodiments, the indicia can be configured to naturally disappear over time (i.e., without the interference of an operator). For example, the indicia can comprise a dissolvable coating configured to dissolve and separate from the device in the presence of saliva. In some embodiments, the indicia can comprise a film adhesively coupled to the device and the film can be separated from the device. In some embodiments, the indicia can be configured to separate from the device when a releasing agent is applied to the indicia and/or the device. Such a releasing agent can include, for example, a chemical, electricity, and/or heat.

### CONCLUSION

Although many of the embodiments are described above with respect to systems, devices, and methods for orthodontic appliances positioned on a lingual side of a patient's teeth, the technology is applicable to other applications and/or other approaches, such as orthodontic appliances positioned on a facial side of the patient's teeth and/or other orthodontic devices. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1-17.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. An orthodontic appliance configured to be secured to a patient's teeth, the appliance comprising:
a resilient member configured to extend along the patient's teeth and to reposition the patient's teeth from an initial position to a desired position, the resilient member comprising one or more securing portions configured to be secured to one or more of the teeth and a marker including indicia configured to communicate information corresponding to at least one of an identity of the patient, an intended position of the appliance relative to the teeth once installed, or an origin of the appliance.

2. The appliance of claim 1, wherein the indicia comprise a recess or opening in the resilient member.

3. The appliance of claim 1, wherein the information corresponding to the origin of the appliance includes a serial number, a stocking keeping unit, and/or a lot number.

4. The appliance of claim 1, wherein the information corresponding to the identity of the patient comprises a combination of at least one of numbers, letters, symbols, shapes, colors, or patterns, and wherein the combination is unique to the patient.

5. The appliance of claim 1, wherein the information corresponding to the intended position of the appliance relative to the teeth indicates which of the patient's teeth the appliance is configured to be secured to.

6. The appliance of claim 1, wherein the indicia include at least one of numbers, letters, symbols, colors, shapes, or patterns.

7. The appliance of claim 1, wherein the indicia is human-readable and/or machine readable.

8. The appliance of claim 1, wherein the resilient member comprises a metal.

9. The appliance of claim 1, wherein the resilient member is formed from a sheet of material.

10. An orthodontic appliance configured to be secured to a patient's teeth, the appliance comprising:
an anchor configured to be disposed along the patient's teeth;
a plurality of arms extending from the anchor, each of the arms including (i) a first end portion extending from the anchor, (ii) a free second end portion, (iii) a connector portion configured to be coupled to one of the teeth, and (iv) a spring portion configured to apply a biasing force to at least one of the teeth, the spring portion disposed along the arm between the first end portion and the connector portion, wherein the anchor and at least some of the arms comprise a three-dimensional unitarily-formed structure; and
a marker configured to communicate information corresponding to at least one of an identity of the patient, an intended position of the appliance relative to the teeth once installed, or an origin of the appliance.

11. The appliance of claim 10, wherein the information corresponding to an intended position of the appliance with respect to the patient's teeth includes an indication of which of the patient's teeth the appliance is configured to be secured to.

12. The appliance of claim 10, wherein the information corresponding to an intended position of the appliance with respect to the patient's teeth includes an indication of which dental arch of the patient the appliance is configured to be secured to.

13. The appliance of claim 10, wherein the origin of the appliance includes a serial number, a stocking keeping unit, and/or a lot number.

14. The appliance of claim 10, wherein the information regarding the identity of the patient comprises a combination of at least one of numbers, letters, symbols, shapes, colors, or patterns, and wherein the combination is unique to the patient.

15. The appliance of claim 10, wherein the marker comprises at least one of a number, a letter, a symbol, a color, a shape, or a pattern.

16. The appliance of claim 10, wherein the marker comprises at least one of a protrusion, a recess, or an opening.

17. The appliance of claim 10, wherein the marker comprises at least one of an ink, a film, a coating, or a surface treatment of the appliance.

18. The appliance of claim 10, wherein the marker is positioned at the anchor.

19. The appliance of claim 10, wherein the marker is positioned at one or more of the plurality of arms.

20. The appliance of claim 10, wherein the marker is positioned at a lingual-facing side of the appliance.

21. The appliance of claim 10, wherein the marker is a first marker configured to communicate information corresponding to an intended position of the appliance relative to the teeth once installed, the appliance further comprising a second marker configured to communicate information corresponding to at least one of an identity of the patient or an origin of the appliance.

22. The appliance of claim 21, wherein the second marker is spaced apart from the first marker.

23. The appliance of claim 10, wherein the marker is permanent.

24. The appliance of claim 10, wherein, when the appliance is secured to the patient's teeth, the marker is not visible within an oral cavity of the patient to a person directly viewing the patient's oral cavity.

25. A method of manufacturing an orthodontic appliance configured to be secured to a patient's teeth, the method comprising:
cutting a substantially planar member from a sheet of material;
while cutting the member from the sheet of material, removing material from the member to form indicia configured to communicate information corresponding to at least one of an origin of the appliance, an identity of the patient, or an instruction for installing the appliance;
manipulating the member into a 3D configuration; and
shape-setting the member in the 3D configuration, thereby forming the orthodontic appliance for installation.

26. The method of claim 25, wherein the one or more recesses extend completely through a thickness of the appliance.

27. The method of claim 25, wherein the indicia includes a combination of at least one of numbers, letters, symbols, colors, shapes, or patterns.

28. The method of claim 25, wherein the indicia is machine-readable.
